(19) United States
(12) Patent Application Publication
(10) Pub. No.: US 2015/0258041 A1
(43) Pub. Date: Sep. 17, 2015

(54) TREATING OSTEOPENIA AND RELATED DISORDERS WITH GERANYLGERANYL ACETONE AND DERIVATIVES THEREOF

(71) Applicant: COYOTE PHARMACEUTICALS, INC., Menlo Park, CA (US)

(72) Inventors: William Boyle, Malibu, CA (US); Naoki Nakayama, Menlo Park, CA (US)

(73) Assignee: COYOTE PHARMACEUTICALS, INC., MENLO PARK, CA (US)

(21) Appl. No.: 14/539,816

(22) Filed: Nov. 12, 2014

Related U.S. Application Data
(63) Continuation of application No. 13/815,792, filed on Mar. 15, 2013, now abandoned.

(51) Int. Cl. A61K 31/121

(52) U.S. Cl. CPC A61K 31/121

(57) ABSTRACT

Provide herein are methods for treating osteopenia with geranylgeranyl acetone (GGA) and derivatives thereof and compositions useful for the same.

### CROSS-REFERENCE TO RELATED APPLICATION

This application is a continuation of U.S. patent application Ser. No. 13/815,792, filed Mar. 15, 2013, which is incorporated herein by reference in its entirety.

### FIELD OF THE INVENTION

This invention provides methods for treating osteopenia including osteoporosis with geranylgeranyl acetone (GGA) and derivatives thereof and compositions useful for the same. Preferably, GGA or the GGA derivative is enriched in the all trans isomer, compared to the relative amount of the trans isomer in the mixtures of cis and trans isomers of GGA or the GGA derivative.

### STATE OF THE ART

Geranylgeranyl acetone (GGA) has the formula:

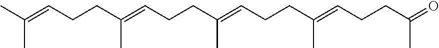 [SMILES: CC(=O)CC/C=C(\C)CC/C=C(\C)CC/C=C(\C)CCC=C(C)C]

and is reported to have neuroprotective and related effects. See, for example, PCT Pat. App. Pub. No. WO 2012/031028 and PCT Pat. App. No. PCT/US2012/027147, each of which is incorporated herein by reference in its entirety.

### SUMMARY OF THE INVENTION

This invention provides methods for treating osteopenia including osteoporosis with geranylgeranyl acetone (GGA) and derivatives thereof and compositions useful for the same. In one aspect, this invention provides methods for treating osteopenia or reducing the negative effects of bone loss comprising administering to a subject in need thereof a therapeutically effective amount of GGA or a GGA derivative. As used herein, subject or patient refers to a mammal, particularly preferably humans.

In some embodiments, treating osteopenia includes without limitation, modulating osteoclast and/or osteoblast function, and preferably, decreasing osteoclast function in diseases such as osteoporosis, hypercalcemia of malignancy, cancer metastasis to the bone, arthritis, Rheumatoid arthritis, bone loss due to immobilization, Paget's disease of the bone, bone loss due to hyperparathyroidism and other metabolic diseases, bone loss due to treatment with corticosteroids, bone loss due to treatment with aromatase inhibitors, periodontal disease, prosthetic loosening and the like. In some embodiments, treating osteopenia includes treating osteoporosis.

In another aspect, this invention provides methods for decreasing osteoclast activity and decreasing bone resorption comprising contacting an osteoclast with an effective amount of GGA or a GGA derivative. In another aspect, this invention provides methods for shifting the balance between osteoclast and osteoblast activity comprising contacting an osteoclast and/or osteoblast with an effective amount of GGA or GGA derivative. In one embodiment, the method further comprises decreasing osteoclast activity and/or increasing osteoblast activity, and/or decreasing bone resorption. In another aspect, this invention provides a method of blocking osteoclast differentiation and/or osteoclast activation of bone resorption, the method comprising contacting an osteoclast with an effective amount of GGA or a GGA derivative.

In another aspect, this invention provides a method for inhibiting loss of bone density in a patient in need thereof comprising administering to the patient an effective amount of GGA or a GGA derivative. In another aspect, this invention provides a method for inhibiting bone fracture in a patient at risk thereof which bone fracture arises at least in part from pathological bone loss comprising administering to the patient an effective amount of GGA or a GGA derivative. In one embodiment, the bone fracture is fracture of the hip. In one embodiment, the bone fracture is fracture of the vertebrae.

In another aspect, this invention provides a method for inhibiting bone loss and/or facilitating bone growth in a patient at a risk of loss of bone density, comprising administering to the patient an effective amount of GGA or a GGA derivative. Without being bound by theory, it is contemplated that the methods and compositions provided herein can increase bone formation and/or reduce bone resorption.

In another aspect, this invention provides methods for treating a subject who undergoes or has undergone a bone grafting procedure, where the bone grafting procedure is autologous (with bone harvested from the patient's own body) includes an allograft (with cadaveric bone usually obtained from a bone bank), or a synthetic graft. The methods described herein can be used to treat a subject prior to, during and/or after a bone grafting procedure.

Preferably, the GGA or the GGA derivative includes the all-trans (hereinafter “trans”) form or substantially the trans form of the GGA or the GGA derivative. As used herein, “substantially” in the context of cis/trans configurations refers to at least 80%, more preferably at least 90%, yet more preferably at least 95%, and most preferably at least 99% of the desired configuration, which can include at least 80%, more preferably at least 90%, yet more preferably at least 95%, and most preferably at least 99% of the trans isomer. In certain embodiments, at least 90%, more preferably, at least 95%, yet more preferably at least 99%, and most preferably, at least 99.5% of the GGA or the GGA derivative is present as a trans isomer.

### DETAILED DESCRIPTION OF THE INVENTION

### Definitions

It must be noted that as used herein and in the appended claims, the singular forms “a”, “an”, and “the” include plural referents unless the context clearly dictates otherwise. Thus, for example, reference to “a solvent” includes a plurality of such solvents.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition or process consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques.

As used herein, Cₘ-Cₙ, such as C₁-C₁₀, C₁-C₆, or C₁-C₄ when used before a group refers to that group containing m to n carbon atoms.

The term “about” when used before a numerical designation, e.g., temperature, time, amount, and concentration, including range, indicates approximations which may vary by (+) or (−) 10%, 5% or 1%.

The term “alkoxy” refers to —O-alkyl.

The term “nitro” refers to —NO₂.

The term “cyano” refers to —CN.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “alkenyl” refers to monovalent aliphatic hydrocarbyl groups having from 2 to 25 carbon atoms or 2 to 6 carbon atoms and 1 or more, preferably 1, carbon carbon double bond. Examples of alkenyl include vinyl, allyl, dimethyl allyl, and the like.

The term “alkynyl” refers to monovalent aliphatic hydrocarbyl groups having from 2 to 25 carbon atoms or 2 to 6 carbon atoms and 1 or more, preferably 1, carbon carbon triple bond.

The term “acyl” refers to —C(O)-alkyl, where alkyl is as defined above.

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

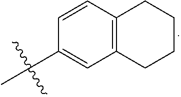 [SMILES: *c1ccc2c(c1)CCCC2]

The term “—CO₂H ester” refers to an ester formed between the —CO₂H group and an alcohol, preferably an aliphatic alcohol. A preferred example included —CO₂R^{E}, wherein R^{E} is alkyl or aryl group optionally substituted with an amino group.

The term “chiral moiety” refers to a moiety that is chiral. Such a moiety can possess one or more asymmetric centers. Preferably, the chiral moiety is enantiomerically enriched, and more preferably a single enantiomer. Non limiting examples of chiral moieties include chiral carboxylic acids, chiral amines, chiral amino acids, such as the naturally occurring amino acids, chiral alcohols including chiral steroids, and the likes.

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

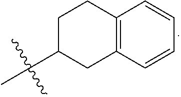 [SMILES: *C1CCc2ccccc2C1]

The term “halo” refers to F, Cl, Br, and/or I.

The term “heteroaryl” refers to a monovalent, aromatic mono-, bi-, or tricyclic ring having 2-14 ring carbon atoms and 1-6 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

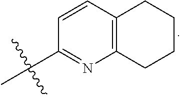 [SMILES: *c1ccc2c(n1)CCCC2]

The term “heterocyclyl” or heterocycle refers to a non-aromatic, mono-, bi-, or tricyclic ring containing 2-10 ring carbon atoms and 1-6 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that they ring is non-aromatic. Nonlimiting examples of heterocyclyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

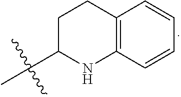 [SMILES: *C1CCc2ccccc2N1]

The term “hydrolyzing” refers to breaking an R^{H}—O—CO—, R^{H}—O—CS—, or an R^{H}—O—SO₂— moiety to an R^{H}—OH, preferably by adding water across the broken bond. A hydrolyzing is performed using various methods well known to the skilled artisan, non limiting examples of which include acidic and basic hydrolysis.

The term “oxo” refers to a C═O group, and to a substitution of 2 geminal hydrogen atoms with a C═O group.

The term “pharmaceutically acceptable” refers to safe and non-toxic for in vivo, preferably, human administration.

The term “pharmaceutically acceptable salt” refers to a salt that is pharmaceutically acceptable.

The term “salt” refers to an ionic compound formed between an acid and a base. When the compound provided herein contains an acidic functionality, such salts include, without limitation, alkai metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionally, such salts include, without limitation, salts of organic acids, such as caroboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisalfate, mono-, di-, and tribasic phosphate, mesylate, tosylate, and the likes.

The term “substantially pure trans isomer” refers to a trans isomer that is by molar amount 95%, preferably 96%, more preferably 99%, and still more preferably 99.5% or more a trans isomer with the rest being the corresponding cis isomer.

“Trans” in the context of GGA and GGA derivatives refer to the GGA scaffold as illustrated below:

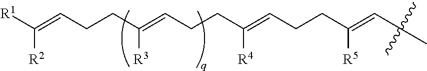

wherein R¹-R⁵ is defined herein and q is 0-2. As shown, each double bond is in a trans or E configuration. In contrast, a cis form of GGA or a GGA derivative will contain one or more of these bonds in a cis or Z configuration.

The term “osteopenia” refers to a disease where osteoclasts resorb more bone than produced by the bone forming cells, osteoblasts. As used herein, treating osteopenias includes without limitation, modulating osteoclast and/or osteoblast function, and preferably, decreasing osteoclast function in diseases such as osteoporosis, hypercalcemia of malignancy, cancer metastasis to the bone, arthritis, Rheumatoid arthritis, bone loss due to immobilization, Paget's disease of the bone, bone loss due to hyperparathyroidism and other metabolic diseases, bone loss due to treatment with corticosteroids, bone loss due to treatment with aromatase inhibitors, periodontal disease, prosthetic loosening and the like. Methods for modulating and or inhibiting osteoclast function are well known to the skilled artisan, and described, for example, in Boyle et al., EP1717315 A3. There are multiple osteoclast culture systems or methods and bone formation assays that can be used successfully to screen potential an anti-resorptive compound of this invention. See, e.g., U.S. Pat. No. 6,080,779.

### GGA Derivatives

GGA derivatives useful in this invention include those described in PCT publication no. WO 2012/031028 and PCT application no. PCT/US2012/027147, each of which are incorporated herein by reference in its entirety. These and other GGA derivatives provided and/or utilized herein are structurally shown below.

In one aspect, the GGA derivative provided and/or utilized herein is of Formula I:

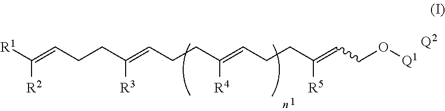

or a tautomer or pharmaceutically acceptable salt thereof, wherein
n¹ is 1 or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;

### Q¹ is —(C═O)—, —(C═S)—, or —S(O₂)—;

Q₂ is hydrogen, R⁶, —O—R⁶, —NR⁷R⁸, or is a chiral moiety;

### R⁶ is:

C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀ aryl, C₂-C₁₀ heteroaryl, wherein each R independently is hydrogen or C₁-C₆ alkyl;

CO— C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹, each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, where R¹⁸ and R¹⁹ together with the nitrogen atom they are attached to form a 5-7 membered heterocycle;
each R⁷ and R⁸ are independently hydrogen or defined as R⁶; and [symbol]
refers to a mixture of cis and trans isomers at the corresponding position wherein at least 80% and, preferably, no more than 95% of the compound of Formula (I) is present as a trans isomer.

In one embodiment, the GGA derivative provided and/or utilized is of Formula (I-A):

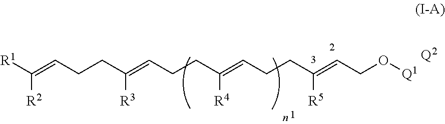

as a substantially pure trans isomer around the 2,3 double bond wherein, n¹, R¹-R⁵, Q¹, and Q² are defined as in Formula (I) above.

In another embodiment, n¹ is 1. In another embodiment, n¹ is 2.

In another embodiment, the GGA derivative provided and/or utilized is of Formula (I-B):

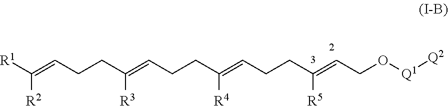

as a substantially pure trans isomer around the 2,3 double bond wherein, R¹-R⁵, Q¹, and Q² are defined as in Formula (I) above.

In another embodiment, the GGA derivative provided and/or utilized is of Formula I-C:

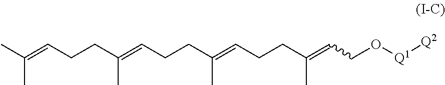

wherein Q¹ and Q² are defined as in Formula (I) above.

In another embodiment, the GGA derivative provided and/or utilized is of Formula (I-D), (I-E), or (I-F):

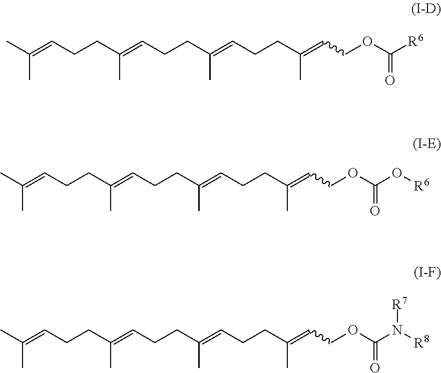

wherein R⁶-R⁸ are defined as in Formula (I) above.

In another embodiment, the GGA derivative provided and/or utilized is of Formula (I-G), (I-H), or (I-I):

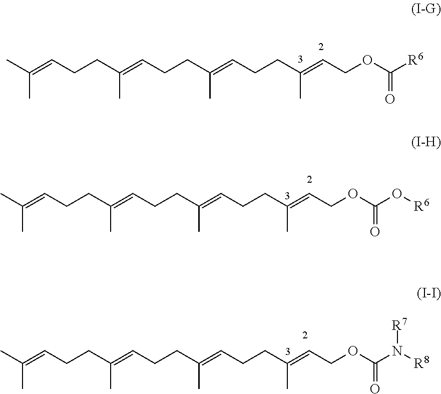

as a substantially pure trans isomer around the 2,3 double bond wherein R⁶-R⁸ are defined as in Formula (I) above.

In a preferred embodiment, R⁶ is C₆-C₁₀ aryl, such as naphthyl. In another preferred embodiment, R⁶ is a heteroaryl, such as quinolinyl.

In another aspect, the GGA derivative provided and/or utilized in this invention is of Formula (II):

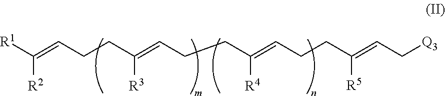

or a pharmaceutically acceptable salt thereof, wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;
Q₃ is —OH, —NR²²R²³—X—CO—NR²⁴R²⁵, —X—CS—NR²⁴R²⁵, or —X—SO₂—NR²⁴R²⁵;

### X is —O—, —S—, —NR²⁶—, or —CR²⁷R²⁸;

each R²² and R²³ independently is hydrogen; C₁-C₆ alkyl, optionally substituted with C₁-C₆ alkoxy; and C₃-C₁₀ cycloalkyl;
each R²⁴ and R²⁵ independently is hydrogen, C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀ aryl, C₂-C₁₀ heteroaryl, wherein each R independently is hydrogen or C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹;
each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, where R¹⁸ and R¹⁹ together with the nitrogen atom they are attached to form a 5-7 membered heterocycle;
R²⁶ is hydrogen or together with R²⁴ or R²⁵ and the intervening atoms form a 5-7 membered heterocyclic ring optionally substituted with 1-3 C₁-C₆ alkyl groups; and
each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered heterocyclyl ring optionally substituted with 1-3 C₁-C₆ alkyl groups.

As used herein, the compound of Formula (II) includes optical isomers such as enantiomers and diastereomers. As also used herein, an ester refers preferably to a phenyl or a C₁-C₆ alkyl ester, which phenyl or alkyl group is optionally substituted with a amino group.

In one embodiment, Q₃ is —NR²²R²³—X—CO—NR²⁴R²⁵, —X—CS—NR²⁴R²⁵, or —X—SO₂—NR²⁴R²⁵. In another embodiment, Q₃ is —X—CO—NR²⁴R²⁵, —X—CS—NR²⁴R²⁵, or —X—SO₂—NR²⁴R²⁵. In another embodiment, Q₃ is —NR²²R²³. In another embodiment, Q₃ is —OH.

In one embodiment, the compound of Formula (II) is of formula:

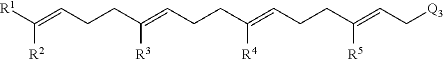

wherein R¹, R², R³, R⁴, R⁵, and Q₃ are defined as in any aspect or embodiment herein.

In another embodiment, the GGA derivative provided and/or utilized is of formula:

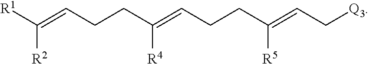

wherein R¹, R², R⁴, R⁵, and Q₃ are defined as in any aspect and embodiment here.

In one embodiment, the compound of Formula (II) is of formula:

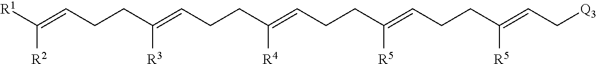

wherein R¹, R², R³, R⁴, R⁵, and Q₃ are defined as in any aspect or embodiment herein.

In another embodiment, the GGA derivative provided and/or utilized is of formula:

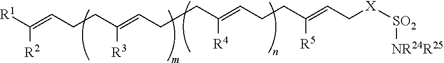

wherein R¹, R², R⁴, R⁵, m, n, X, R²⁴ and R²⁵ are defined as in any aspect and embodiment here.

In another embodiment, the GGA derivative provided and/or utilized is of formula:

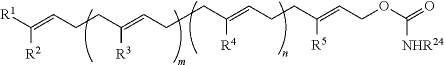

wherein R¹, R², R⁴, R⁵, m, n, and R²⁴ are defined as in any aspect and embodiment here.

In another embodiment, the GGA derivative provided and/or utilized is of formula:

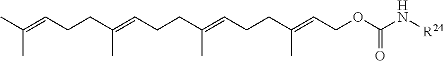

wherein R²⁴ is defined as in any aspect and embodiment here.

In another embodiment, the GGA derivative provided and/or utilized is of formula:

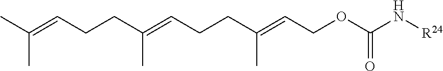

wherein R²⁴ is defined as in any aspect and embodiment here.

In another embodiment, the GGA derivative provided and/or utilized is of formula:

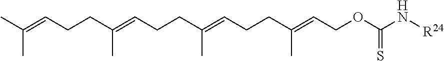

wherein R²⁴ is defined as in any aspect and embodiment here.

In another embodiment, the GGA derivative provided and/or utilized is of formula:

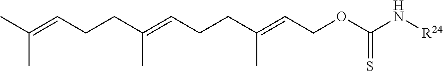

wherein R²⁴ is defined as in any aspect and embodiment here.

In another embodiment, the GGA derivative provided and/or utilized is of formula:

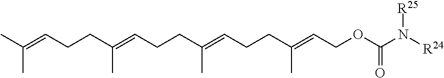

wherein R²⁴ and R²⁵ are defined as in any aspect and embodiment here.

In another embodiment, the GGA derivative provided and/or utilized is of formula:

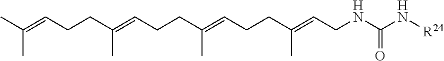

wherein R²⁴ is defined as in any aspect and embodiment here.

In another embodiment, the GGA derivative provided and/or utilized is of formula:

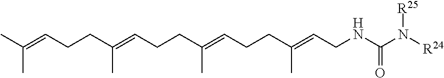

wherein R²⁴ and R²⁵ are defined as in any aspect and embodiment here.

In one embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

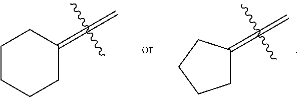 [SMILES: *=C1CCCCC1 ; *=C1CCCC1]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, Q₃ is —X—CO—NR²⁴R²⁵. In another embodiment, Q₃ is —X—CS—NR²⁴R²⁵. In another embodiment, Q₃ is —X—SO₂—NR²⁴R²⁵. In another embodiment, Q₃ is —OCONHR²⁴, —OCONR²⁴R²⁵, —NHCONHR²⁴, —NHCONR²⁴R²⁵, —OCSNHR²⁴, —OCSNR²⁴R²⁵, —NHCSNHR²⁴, or —NHCSNR²⁴R²⁵.

In another embodiment, X is —O—. In another embodiment, X is —NR²⁶—. In another embodiment, X is or —CR²⁷R²⁸.

In another embodiment, one of R²⁴ and R²⁵ is hydrogen. In another embodiment, one or both of R²⁴ and R²⁵ are C₁-C₆ alkyl. In another embodiment, one or both of R²⁴ and R²⁵ are C₁-C₆ alkyl, optionally substituted with an R² group, wherein R²⁰ is —CO₂H or an ester thereof, C₁-C₆ alkyl, C₃-C₁₀ cycloalkyl, C₃-C₈ heterocyclyl, C₆-C₁₀ aryl, or C₂-C₁₀ heteroaryl. In another embodiment, one or both of R²⁴ and R²⁵ are C₃-C₁₀ cycloalkyl. In another embodiment, one or both of R²⁴ and R²⁵ are C₃-C₁₀ cycloalkyl substituted with 1-3 alkyl groups. In another embodiment, one or both of R²⁴ and R²⁵ are C₃-C₈ heterocyclyl. In another embodiment, one or both of R²⁴ and R²⁵ are C₆-C₁₀ aryl. In another embodiment, one or both of R²⁴ and R²⁵ are C₂-C₁₀ heteroaryl. In another embodiment, R²⁴ and R²⁵ together with the nitrogen atom they are attached to form a 5-7 membered heterocycle.

In another embodiment, R²⁰ is —CO₂H or an ester thereof. In another embodiment, R²⁰ is C₁-C₆ alkyl. In another embodiment, R²⁰ is C₃-C₁₀ cycloalkyl. In another embodiment, R²⁰ is C₃-C₈ heterocyclyl. In another embodiment, R²⁰ is C₆-C₁₀ aryl. In another embodiment, R²⁰ is or C₂-C₁₀ heteroaryl.

In another embodiment, the GGA derivative provided and/or utilized is of formula (II):

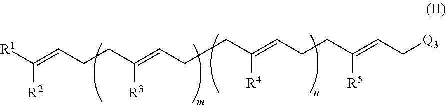

or a pharmaceutically acceptable salt thereof, wherein
   m is 0 or 1;
   n is 0, 1, or 2;
   each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
   each of R³, R⁴, and R⁵ independently are hydrogen or C₁-C₆ alkyl;
   Q₃ is —X—CO—NR²⁴R²⁵ or —X—SO₂—NR²⁴R²⁵;
   X is —O—, —NR²⁶—, or —CR²⁷R²⁸;
   R²⁶ is hydrogen or together with R²⁴ or R²⁵ and the intervening atoms form a 5-7 membered ring optionally substituted with 1-3 C₁-C₆ alkyl groups;
   each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered cycloalkyl or heterocyclyl ring optionally substituted with 1-3 C₁-C₆ alkyl groups;
   each R²⁴ and R²⁵ independently is
   hydrogen,
   C₁-C₆ alkyl, optionally substituted with —CO₂H or an ester thereof, C₃-C₁₀ preferably C₃-C₈ cycloalkyl, C₃-C₈ heterocyclyl, C₆-C₁₀ aryl, or C₂-C₁₀ heteroaryl,
   C₃-C₁₀ cycloalkyl,
   C₃-C₈ heterocyclyl,
   C₆-C₁₀ aryl, or
   C₂-C₁₀ heteroaryl,
wherein each cycloalkyl, heterocyclyl, aryl, or heteroaryl is optionally substituted with 1-3 C₁-C₆ alkyl groups, or R²⁴ and R²⁵ together with the nitrogen atom they are attached to form a 5-7 membered heterocycle.

In another embodiment, provided herein are compounds of formula:

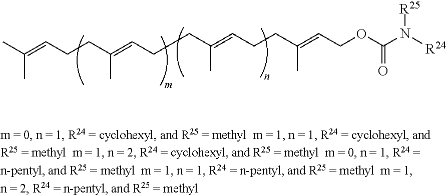

In another aspect, the GGA derivative provided and/or utilized herein is of Formula III:

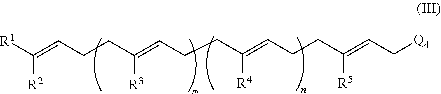

or a pharmaceutically acceptable salt of each thereof, wherein

m is 0 or 1;

n is 0, 1, or 2;

each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;

each of R³, R⁴, and R⁵ independently are hydrogen or C₁-C₆ alkyl;

Q₄ is selected from the group consisting of:

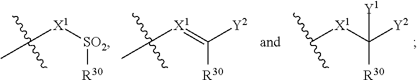

when X¹ is bonded via a single bond, X¹ is —O—, —NR³¹—, or —CR³²R³³—, and when X¹ is bonded via a double bond, X¹ is —CR³²—;

Y¹ is hydrogen, —OH or —O—R¹⁰, Y² is —OH, —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹⁷);

R³⁰ is C₁-C₆ alkyl optionally substituted with 1-3 alkoxy or 1-5 halo group, C₂-C₆ alkenyl, C₂-C₆ alkynyl, C₃-C₁₀ cycloalkyl, C₆-C₁₀ aryl, C₃-C₈ heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups, or R³⁰ is —NR²⁴R³⁵;

R³¹ is hydrogen or together with R³⁰ and the intervening atoms form a 5-7 membered ring optionally substituted with 1-3 C₁-C₆ alkyl groups;

each R³² and R³³ independently are hydrogen, C₁-C₆ alkyl, —COR⁸¹ or —CO₂R⁸¹, or R³² together with R³⁰ and the intervening atoms form a 5-7 membered cycloalkyl or heterocyclyl ring optionally substituted with oxo or 1-3 C₁-C₆ alkyl groups;

R¹⁰ is C₁-C₆ alkyl;

R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a heterocycle optionally substituted with 1-3 C₁-C₆ alkyl groups;

R¹³ is C₁-C₆ alkyl or C₃-C₁₀ cycloalkyl optionally substituted with 1-3 C₁-C₆ alkyl groups;

R¹⁴ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;
each R¹⁵ independently are hydrogen, C₃-C₁₀ cycloalkyl, C₁-C₆ alkyl optionally substituted with 1-3 substituents selected from the group consisting of —CO₂H or an ester thereof, aryl, or C₃-C₈ heterocyclyl, or two R¹⁵ groups together with the nitrogen atom they are bonded to form a 5-7 membered heterocycle;

R¹⁶ is hydrogen or C₁-C₆ alkyl;

R¹⁷ is hydrogen, C₁-C₆ alkyl substituted with 1-3 hydroxy groups, —CHO, or is CO₂H or an ester thereof;

each R³⁴ and R³⁵ independently is hydrogen, C₁-C₆ alkyl, optionally substituted with —CO₂H or an ester thereof, C₃-C₁₀ cycloalkyl, C₃-C₈ heterocyclyl, C₆-C₁₀ aryl, or C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R³⁴ and R³⁵ together with the nitrogen atom they are attached to form a 5-7 membered heterocycle; and

each R⁸¹ independently is C₁-C₆ alkyl.

In one embodiment, m is 0. In another embodiment, m is 1. In another embodiment, n is 0. In another embodiment, n is 1. In another embodiment, n is 2.

In one embodiment, the compound of Formula (III) is of formula:

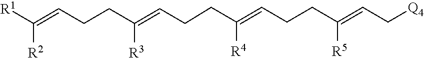

wherein Q₄, R¹, R², R³, R⁴, R⁵, R³⁰, X¹, Y¹, and Y² are defined as in any aspect or embodiment herein.

In one embodiment, the GGA derivative provided and/or utilized is of formula:

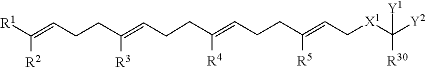

wherein R¹, R², R³, R⁴, R⁵, R³⁰, X¹, Y¹, and Y² are defined as in any aspect and embodiment here.

In another embodiment, the GGA derivative provided and/or utilized is of formula:

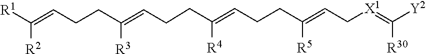

wherein R¹, R², R³, R⁴, R⁵, R³⁰, X¹, and Y² are defined as in any aspect and embodiment herein.

In another embodiment, the GGA derivative provided and/or utilized is of formula:

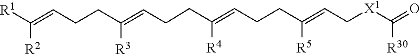

wherein R¹, R², R³, R⁴, R⁵, R³⁰ and X¹ are defined as in any aspect and embodiment herein.

In another embodiment, the GGA derivative provided and/or utilized is of formula:

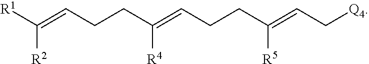

wherein R¹, R², R⁴, R⁵, and Q₄ are defined as in any aspect and embodiment herein.

In another embodiment, the GGA derivative provided and/or utilized is of formula:

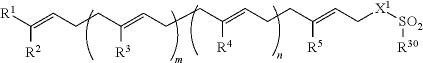

wherein R¹, R², R⁴, R⁵, m, n, X¹, and R³⁰ are defined as in any aspect and embodiment here.

In another embodiment, the GGA derivative provided and/or utilized is of formula:

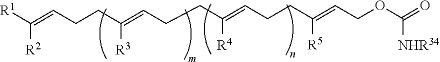

wherein R¹, R², R⁴, R⁵, m, n, and R³⁴ are defined as in any aspect and embodiment here.

In another embodiment, the GGA derivative provided and/or utilized is of formula:

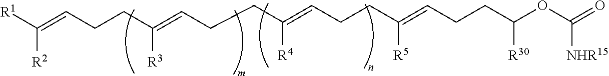

wherein R¹, R², R⁴, R⁵, R³⁰, m, n, and R¹⁵ are defined as in any aspect and embodiment here.

In another embodiment, each R¹ and R² are C₁-C₆ alkyl. In another embodiment, each R¹ and R² are methyl, ethyl, or isopropyl. In another embodiment, R¹ and R² together with the carbon atom they are attached to form a 5-6 membered ring optionally substituted with 1-3 C₁-C₆ alkyl groups. In another embodiment, R¹ and R² together with the carbon atom they are attached to form a ring that is:

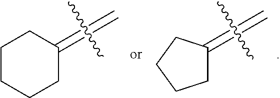

In another embodiment, R³, R⁴, and R⁵ are C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, X¹ is O. In another embodiment, X¹ is —NR¹³. In another embodiment, R³¹ is hydrogen. In another embodiment, R³¹ together with R³⁰ and the intervening atoms form a 5-7 membered ring optionally substituted with 1-3 C₁-C₆ alkyl groups. In another embodiment, X¹ is —CR³²R³³—. In another embodiment, X¹ is —CR³²—. In another embodiment, each R³² and R³³ independently are hydrogen, C₁-C₆ alkyl, —COR⁸¹, or —CO₂R⁸¹. In another embodiment, R³² is hydrogen, and R³³ is hydrogen, C₁-C₆ alkyl, —COR⁸¹, or —CO₂R⁸¹.

In another embodiment, R³³ is hydrogen. In another embodiment, R³³C₁-C₆ alkyl. In another embodiment, R³³ is methyl. In another embodiment, R³³ is —CO₂R⁸¹. In another embodiment, R³³ is —COR⁸¹.

In another embodiment, R³² together with R³⁰ and the intervening atoms form a 5-7 membered ring. In another embodiment, the moiety:

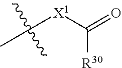

which is “Q₄,” has the structure:

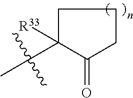

wherein R³³ is hydrogen, C₁-C₆ alkyl, or —CO₂R⁸¹ and n is 1, 2, or 3. Within these embodiments, in certain embodiments, R³³ is hydrogen or C₁-C₆ alkyl. In one embodiment, R³³ is hydrogen. In another embodiment, R³³ is C₁-C₆ alkyl.

In another embodiment, R³⁰ is C₁-C₆ alkyl. In another embodiment, R³⁰ is methyl, ethyl, butyl, isopropyl, or tertiary butyl. In another embodiment, R³⁰ is C₁-C₆ alkyl substituted with 1-3 alkoxy or 1-5 halo group. In another embodiment, R³⁰ is alkyl substituted with an alkoxy group. In another embodiment, R³⁰ is alkyl substituted with 1-5, preferably, 1-3, halo, preferably fluoro, groups.

In another embodiment, R³⁰ is NR³⁴R³⁵. In a preferred embodiment, R³⁵ is H. In a preferred embodiment, R³⁴ is C₁-C₆ alkyl, optionally substituted with a group selected from the group consisting of —CO₂H or an ester thereof, C₃-C₁₀ cycloalkyl, C₃-C₈ heterocyclyl, C₆-C₁₀ aryl, or C₂-C₁₀ heteroaryl. In another preferred embodiment, R³⁴ is C₃-C₁₀ cycloalkyl, C₃-C₈ heterocyclyl, C₆-C₁₀ aryl, or C₂-C₁₀ heteroaryl. In a more preferred embodiment, R³⁴ is C₃-C₁₀ cycloalkyl.

In another embodiment, R³⁰ is C₂-C₆ alkenyl or C₂-C₆ alkynyl. In another embodiment, R³⁰ is C₃-C₁₀ cycloalkyl. In another embodiment, R³⁰ is C₃-C₁₀ cycloalkyl substituted with 1-3 C₁-C₆ alkyl groups. In another embodiment, R³⁰ is cyclopropyl, cyclobutyl, cyclopentyl, cyclohexyl, or adamentyl. In another embodiment, R³ is C₆-C₁₀ aryl or C₂-C₁₀ heteroaryl. In another embodiment, R³⁰ is a 5-7 membered heteroaryl containing at least 1 oxygen atom. In another embodiment, R³⁰ is C₆-C₁₀ aryl, C₃-C₈ heterocyclyl, or C₂-C₁₀ heteroaryl, wherein each aryl, heterocyclyl, or heteroaryl is optionally substituted with 1-3 C₁-C₆ alkyl groups.

In another embodiment, Y² is —O—R¹¹. In another embodiment, Y¹ and Y² are joined to form ═NR¹³. In another embodiment, Y¹ and Y² are joined to form ═NOR¹⁴. In another embodiment, Y¹ and Y² are joined to form (═O). In another embodiment, Y¹ and Y² are joined to form ═CR¹⁶R¹⁷.

In another embodiment, Q₄ is —CR³³COR³⁰. In another embodiment, R³⁰ is C₁-C₆ alkyl optionally substituted with an alkoxy group. In another embodiment, R³⁰ is C₃-C₈ cycloalkyl. In another embodiment, R³³ is hydrogen. In another embodiment, R³³ is C₁-C₆ alkyl. In another embodiment, R³³ is CO₂R⁸¹. In another embodiment, R³³ is COR⁸¹.

In another embodiment, Q₄ is —CH₂—CH(O—CONHR¹⁵)—R³⁰. In another embodiment, R¹⁵ is C₃-C₈ cycloalkyl. In another embodiment, R¹⁵ is C₁-C₆ alkyl optionally substituted with 1-3 substituents selected from the group consisting of —CO₂H or an ester thereof, aryl, or C₃-C₈ heterocyclyl. In a preferred embodiment within these embodiments, R³⁰ is C₁-C₆ alkyl.

In another embodiment, Q₄ is —O—CO—NHR³⁴. withing these embodiment, in another embodiment, R³⁴ is C₁-C₆ alkyl, optionally substituted with —CO₂H or an ester thereof, C₃-C₈ cycloalkyl, C₃-C₈ heterocyclyl, C₂-C₁₀ aryl, or C₂-C₁₀ heteroaryl. In yet another embodiment, R³⁴ is C₃-C₈ cycloalkyl, C₃-C₈ heterocyclyl, C₂-C₁₀ aryl, or C₂-C₁₀ heteroaryl.

In another embodiment, R¹⁴ is hydrogen. In another embodiment, R¹⁴ is C₁-C₆ alkyl optionally substituted with a —CO₂H or an ester thereof or a C₆-C₁₀ aryl optionally substituted with 1-3 alkyl groups. In another embodiment, R¹⁴ is C₂-C₆ alkenyl. In another embodiment, R¹⁴ is C₂-C₆ alkynyl In another embodiment, R¹⁴ is C₃-C₆ cycloalkyl optionally substituted with 1-3 alkyl groups. In another embodiment, R¹⁴ is C₃-C₈ heterocyclyl optionally substituted with 1-3 alkyl groups.

In another embodiment, preferably, R¹⁶ is hydrogen. In another embodiment, R¹⁷ is CO₂H or an ester thereof. In another embodiment, R¹⁷ is C₁-C₆ alkyl substituted with 1-3 hydroxy groups. In another embodiment, R¹⁷ is C₁-C₃ alkyl substituted with 1 hydroxy group. In another embodiment, R¹⁷ is —CH₂OH.

In another embodiment, R¹⁰ and R¹¹ together with the intervening carbon atom and oxygen atoms form a heterocycle of formula:

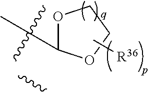

wherein q is 0 or 1, p is 0, 1, 2, or 3, and R³⁶ is C₁-C₆ alkyl.

In another embodiment, q is 1. In another embodiment, q is 2. In another embodiment, p is 0. In another embodiment, p is 1. In another embodiment, p is 2. In another embodiment, p is 3.

In one aspect, the GGA derivative provided and/or utilized herein is of Formula (IV):

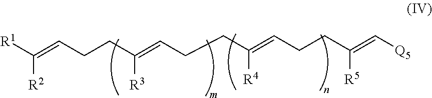

or a tautomer thereof, or a pharmaceutically acceptable salt of each thereof, wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups; each of R³, R⁴, and R⁵ independently are hydrogen or C₁-C₆ alkyl, or R⁵ and Q₅ together with the intervening carbon atoms form a 6 membered aryl ring, or a 5-8 membered cycloalkenyl ring, or a 5-14 membered heteroaryl or heterocycle, wherein each aryl, cycloalkenyl, heteroaryl, or heterocycle, ring is optionally substituted with 1-2 substituents selected from the group consisting of halo, hydroxy, oxo, —N(R⁴⁰)₂, and C₁-C₆ alkyl group;
Q₅ is —C(═O)H, —CO₂H or —CH═CHCO₂H, or a C₁-C₆ alkyl ester or acyl halide thereof, wherein the ester is optionally substituted with —CO-phenyl; a 6-10 membered aryl or a 5-14 membered heteroaryl or heterocycle containing up to 6 ring heteroatoms, wherein the heteroatom is selected from the group consisting of O, N, S, and oxidized forms of N and S, and further wherein the aryl, heteroaryl, or heterocyclyl ring is optionally substituted with 1-3 substituents selected from the group consisting of:

hydroxy, oxo, —N(R⁴⁰)₂, C₁-C₆ alkoxy group, and C₁-C₆ alkyl group,

wherein the alkyl group is optionally substituted with 1-3 substituents selected from hydroxy, NH₂, C₆-C₁₀ aryl, —CO₂H or an ester or an amide thereof,

a 5-9 membered heteroaryl containing up to 3 ring heteroatoms, wherein the heteroaryl is optionally substituted with 1-3 hydroxy, —N(R⁴⁰)₂, and C₁-C₆ alkyl group,

benzyl, and phenyl optionally substituted with 1-3 substituents selected from the group consisting of C₁-C₆ alkyl, C₁-C₆ alkoxy, hydroxy, and halo groups; and wherein each R⁴⁰ independently is hydrogen or C₁-C₆ alkyl.

As used herein, the compound of Formula (IV) includes tautomers and optical isomers such as enantiomers and diastereomers. As also used herein, an ester refers preferably to a phenyl or a C₁-C₆ alkyl ester, which phenyl or alkyl group is optionally substituted with a amino group. As used herein, an amide refers preferably to a moiety of formula —CON(R⁴⁰)₂, wherein R⁴⁰ is defined as above.

In some embodiment, Q₆ is selected from a group consisting of oxazole, oxadiazole, oxazoline, azalactone, imidazole, diazole, triazole, and thiazole, wherein each heteroaryl or heterocycle is optionally substituted as disclosed above.

In one embodiment, the GGA derivative provided and/or utilized is of formula IV-A:

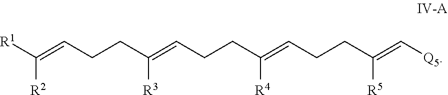

In another embodiment, the GGA derivative provided and/or utilized is of formula IV-B:

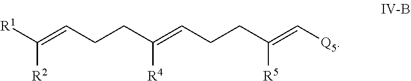

wherein R¹, R², R⁴, R⁵, and Q₅ are defined as in any aspect and embodiment here.

In another embodiment, Q₅ is selected from the group consisting of:

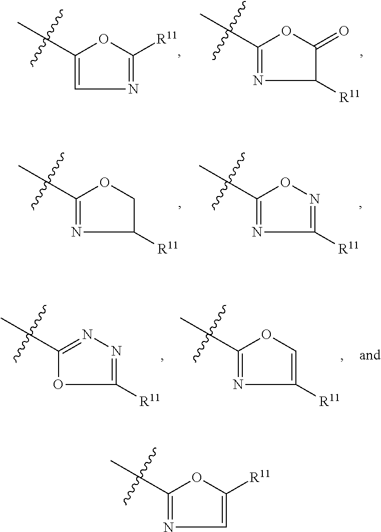

wherein R¹¹ is C₁-C₆ alkyl, C₆-C₁₀ aryl, C₃-C₈ heteroaryl, C₃-C₈ heteroaryl, C₃-C₁₀ cycloalkyl and the alkyl group is optionally substituted with 1-3 C₆-C₁₀ aryl, C₃-C₈ heteroaryl, C₃-C₈ heteroaryl, C₃-C₁₀ cycloalkyl groups, and the aryl, heteroaryl, heteroaryl, cycloalkyl groups are optionally substituted with 1-3 C₁-C₆ alkyl, C₁-C₆ alkoxy, halo, preferably chloro or fluoro, C₆-C₁₀ aryl, C₃-C₈ heteroaryl, C₃-C₈ heteroaryl, C₃-C₁₀ cycloalkyl group.

In another embodiment, Q₅ is phenyl, optionally substituted as described herein. In another embodiment, Q₅ is benzimidazole, benzindazole, and such other 5-6 fused 9-membered bicyclic heteroaryl or heterocycle. In another embodiment, Q₅ is quinoline, isoquinoline, and such other 6-6 fused 10 membered heteroaryl or heterocycle. In another embodiment, Q₅ is benzodiazepine or a derivative thereof, such as, a benzodiazepinone. Various benzodiazepine and derivatives thereof are well known to the skilled artisan.

In another embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

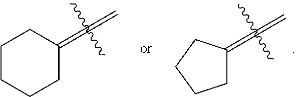 [SMILES: *=C1CCCCC1 ; *=C1CCCC1]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, this invention provides a compound selected from the group consisting of:

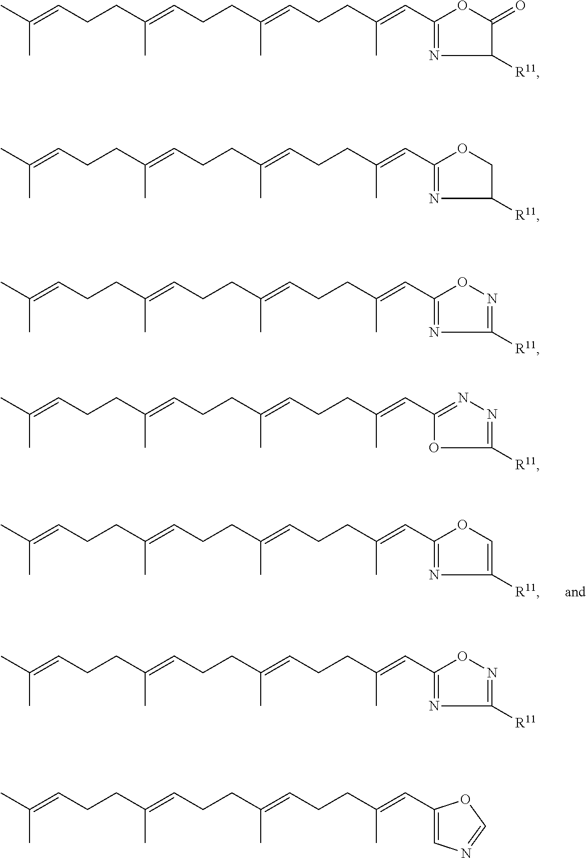

wherein R¹¹ is defined as above.

In another aspect, GGA derivatives provided and/or utilized herein are of formula (V):

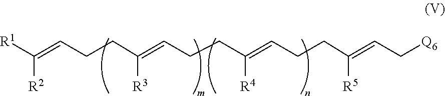

or a pharmaceutically acceptable salt thereof, wherein
m is 0 or 1;
n is 0, 1, or 2;
each R¹ and R² independently are C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently is hydrogen or C₁-C₆ alkyl;
Q₆ is selected from the group consisting of:

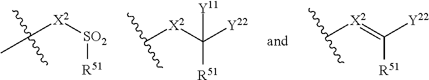

when X² is bonded via a single bond, X² is —O—, —NR⁵²—, or —CR⁵³R⁵⁴—, and when X² is bonded via a double bond, X² is —CR⁵³—;
Y¹¹ is hydrogen, —OH or —OR⁵⁵;
Y²² is —OH, —OR⁵⁶, —NHR⁵⁷, or —O—CO—NR⁵⁸R⁵⁹, or Y¹¹ and Y²² are joined to form an oxo group (═O), an imine group (═NR⁶⁰), a oxime group (═N—OR⁶¹), or a substituted or unsubstituted vinylidene (═CR⁶³R⁶⁴);
R⁵¹ is C₁-C₆ alkyl, C₁-C₆ alkyl substituted with 1-3 alkoxy or 1-5 halo groups, C₂-C₆ alkenyl, C₂-C₆ alkynyl, C₃-C₁₀ cycloalkyl, C₃-C₈ heterocyclyl, C₆-C₁₀ aryl, C₂-C₁₀ heteroaryl, or —NR⁶⁵R⁶⁶, wherein each cycloalkyl or heterocyclyl is optionally substituted with 1-3 C₁-C₆ alkyl groups, and wherein each aryl or heteroaryl is optionally substituted independently with 1-3 nitro and C₁-C₆ alkyl groups;

R⁵² is hydrogen or together with R⁵¹ and the intervening atoms form a 5-7 membered ring optionally substituted with 1-3 C₁-C₆ alkyl groups;
each R⁵³ and R⁵⁴ independently are hydrogen, C₁-C₆ alkyl, —COR⁸¹, —CO₂R⁸¹, or —CONHR⁸², or R⁵³ together with R⁵¹ and the intervening atoms form a 5-7 membered cycloalkyl or heterocyclyl ring optionally substituted with 1-3 C₁-C₆ alkyl groups;
R⁵⁵ is C₁-C₆ alkyl;
each R⁵⁶ and R⁵⁷ independently are C₁-C₆ alkyl, C₃-C₁₀ cycloalkyl, —CO₂R⁶², or —CON(R⁶²)₂; or R⁵⁵ and R⁵⁶ together with the intervening carbon atom and oxygen atoms form a heterocycle optionally substituted with 1-3 C₁-C₆ alkyl groups;
R⁵⁸ is: C₃-C₁₀ cycloalkyl, C₁-C₆ alkyl optionally substituted with —OH, CO₂H or an ester thereof, or C₃-C₁₀ cycloalkyl,

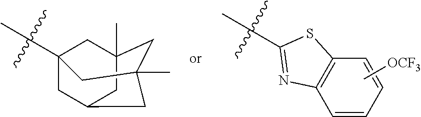

R⁵⁹ is hydrogen or C₁-C₆ alkyl;
R⁶⁰ is C₁-C₆ alkyl or C₃-C₁₀ cycloalkyl optionally substituted with 1-3 C₁-C₆ alkyl groups, or is:

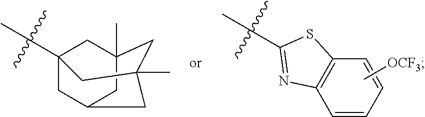

R⁶¹ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;
each R⁶² independently are hydrogen, C₃-C₁₀ cycloalkyl, C₁-C₆ alkyl optionally substituted with 1-3 substituents selected from the group consisting of —CO₂H or an ester thereof, aryl, C₃-C₈ heterocyclyl, or two R⁶² groups together with the nitrogen atom they are bonded to form a 5-7 membered heterocycle;
R⁶³ is hydrogen or C₁-C₆ alkyl;
R⁶⁴ is hydrogen, C₁-C₆ alkyl substituted with 1-3 hydroxy groups, —CHO, or is CO₂H or an ester thereof;
one or both of R⁶⁵ and R⁶⁶ independently are hydrogen, C₁-C₆ alkyl, optionally substituted with —CO₂H or an ester thereof, C₃-C₁₀ cycloalkyl, C₃-C₈ heterocyclyl, C₂-C₁₀ aryl, or C₂-C₁₀ heteroaryl, or is C₃-C₁₀ cycloalkyl, C₃-C₈ heterocyclyl, C₆-C₁₀aryl, or C₂-C₁₀ heteroaryl, wherein each cycloalkyl, heterocyclyl, aryl, or heteroaryl is optionally substituted with 1-3 alkyl groups, or R⁶⁵ and R⁶⁶ gether with the nitrogen atom they are bonded to form a 5-7 membered heterocycle, and if only one of R⁶⁵ and R⁶⁶ are defined as above, then the other one is

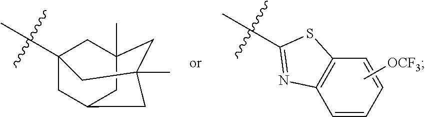

and
R⁸¹ is C₁-C₆ alkyl; and
R⁸² is:

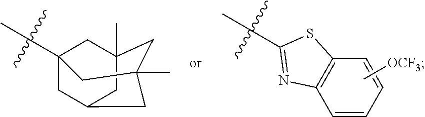

provided that, when X² is bonded via a single bond, and R⁵³ or R⁵⁴ is not —CONHR⁸², Y¹¹ and Y²² are joined to form an imine group (═NR⁶⁰), and R⁶⁰ is: or OCF₃

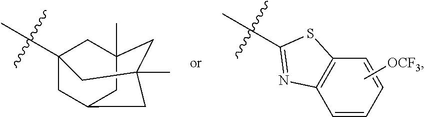

or Y²² is —O—CO—NR⁵⁸R⁵⁹;
or provided that, when Q₆ is:

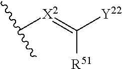

and R⁵³ is not —CONHR⁸², Y²² is —O—CO—NR⁵⁸R⁵⁹;
or provided that, when Q₆ is —O—CO—NR⁶⁵R⁶⁶, then at least one of R⁶⁵ and R⁶⁶ is:

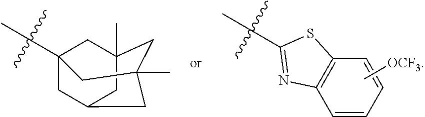

In one embodiment, the GGA derivative provided and/or utilized are of formula:

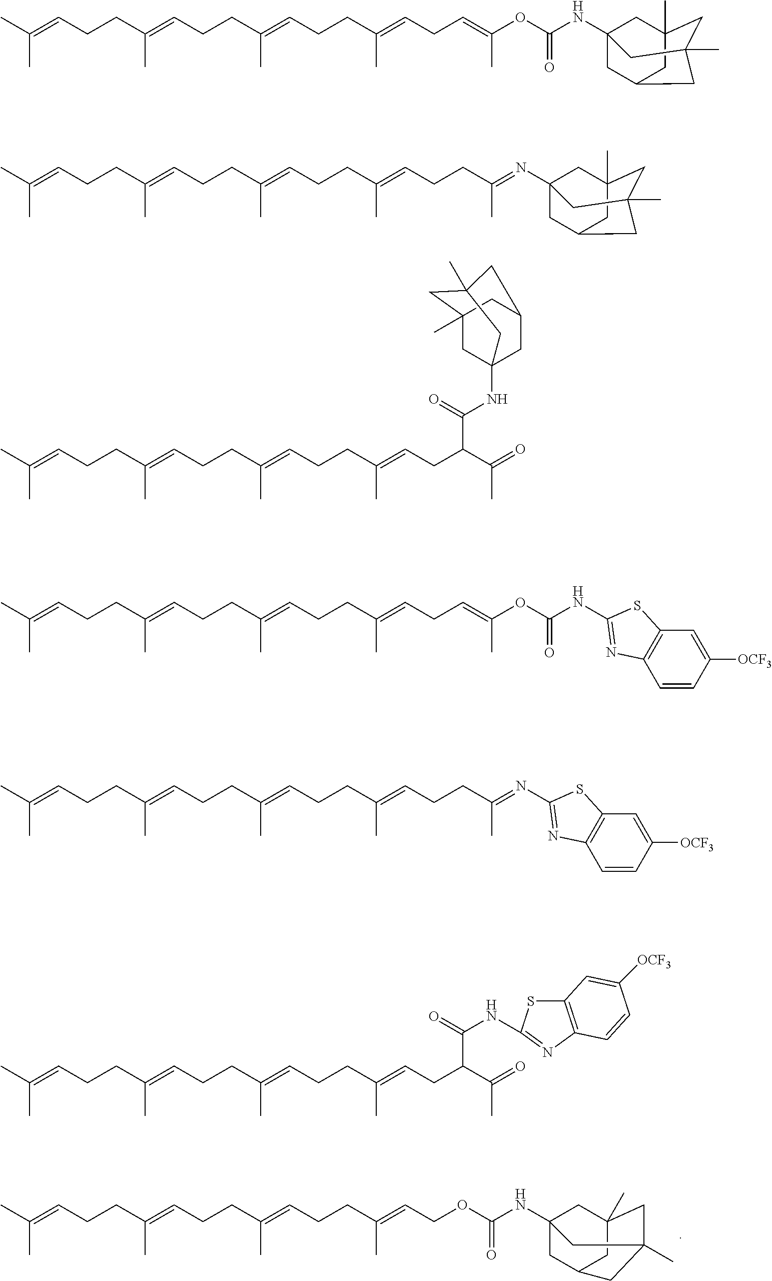

In another aspect, the GGA derivatives useful according to this invention is selected from:

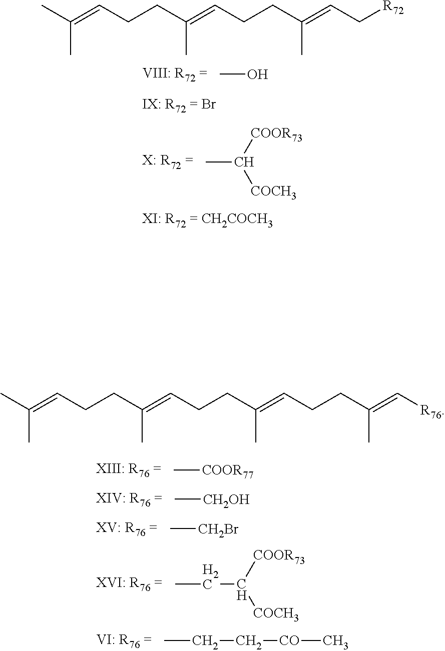

In one embodiment, the compounds provided herein excludes the compound of formula:

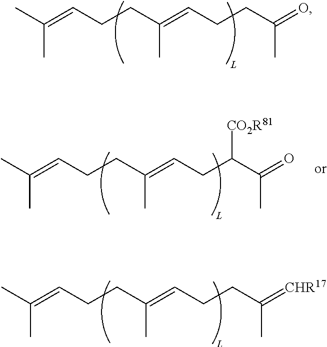

wherein L is 0, 1, 2, or 3, and R¹⁷ is CO₂H or an ester thereof, or is —CH₂OH, or is a C₁-C₆ alkyl ester of —CH₂OH.

In another embodiment, examples of compounds provided and/or utilized by this invention include certain compounds tabulated below. Compound ID numbers in Table I refer to synthetic schemes in Example 7.

TABLE 1

| Compound ID | Structure |
|---|---|
| 1 | 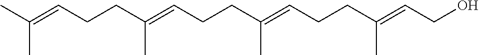 [SMILES: CC(C)=CCC/C(C)=C/CC/C(C)=C/CC/C(C)=C/CO] |
| 2a | 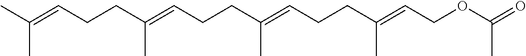 [SMILES: CC(=O)OC/C=C(\C)CC/C=C(\C)CC/C=C(\C)CCC=C(C)C] |
| 2b | 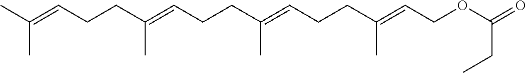 [SMILES: CCC(=O)OC/C=C(\C)CC/C=C(\C)CC/C=C(\C)CCC=C(C)C] |
| 2c | 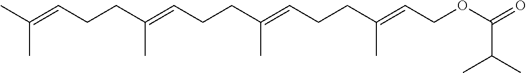 [SMILES: CC(C)=CCC/C(C)=C/CC/C(C)=C/CC/C(C)=C/COC(=O)C(C)C] |
| 2d | 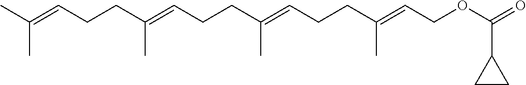 [SMILES: CC(C)=CCC/C(C)=C/CC/C(C)=C/CC/C(C)=C/COC(=O)C1CC1] |
| 2e | 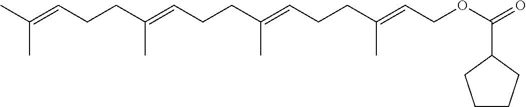 [SMILES: CC(C)=CCC/C(C)=C/CC/C(C)=C/CC/C(C)=C/COC(=O)C1CCCC1] |
| 2f | 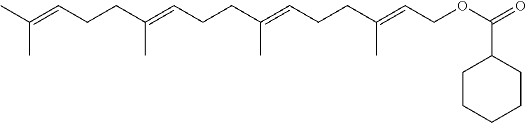 [SMILES: CC(C)=CCC/C(C)=C/CC/C(C)=C/CC/C(C)=C/COC(=O)C1CCCCC1] |
| 2g | 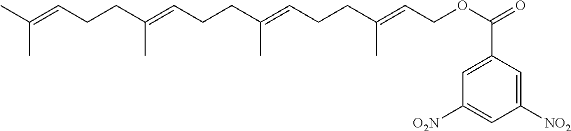 [SMILES: CC(C)=CCC/C(C)=C/CC/C(C)=C/CC/C(C)=C/COC(=O)c1cc([N+](=O)[O-])cc([N+](=O)[O-])c1] |
| 2h | 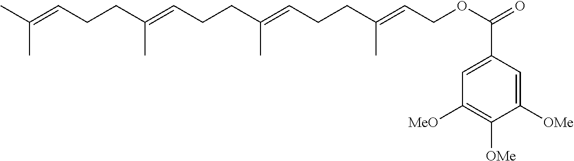 [SMILES: COc1cc(C(=O)OC/C=C(\C)CC/C=C(\C)CC/C=C(\C)CCC=C(C)C)cc(OC)c1OC] |
| 2i | 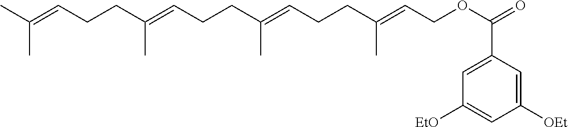 [SMILES: CCOc1cc(OCC)cc(C(=O)OC/C=C(\C)CC/C=C(\C)CC/C=C(\C)CCC=C(C)C)c1] |
| 2j | 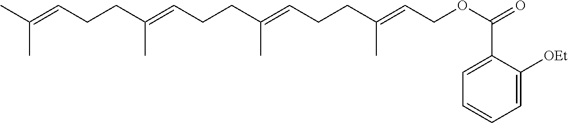 [SMILES: CCOc1ccccc1C(=O)OC/C=C(\C)CC/C=C(\C)CC/C=C(\C)CCC=C(C)C] |
| 2k | 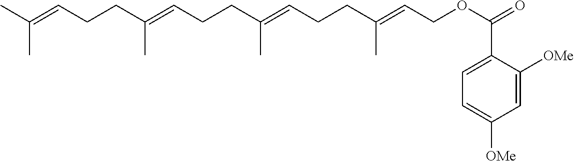 [SMILES: COc1ccc(C(=O)OC/C=C(\C)CC/C=C(\C)CC/C=C(\C)CCC=C(C)C)c(OC)c1] |
| 2l | 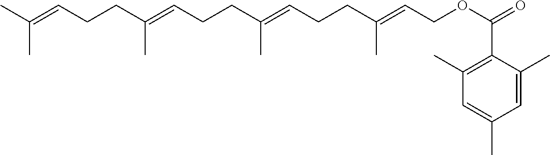 [SMILES: CC(C)=CCC/C(C)=C/CC/C(C)=C/CC/C(C)=C/COC(=O)c1c(C)cc(C)cc1C] |
| 4a | 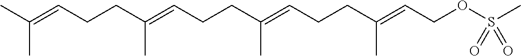 [SMILES: CC(C)=CCC/C(C)=C/CC/C(C)=C/CC/C(C)=C/COS(C)(=O)=O] |
| 4b | 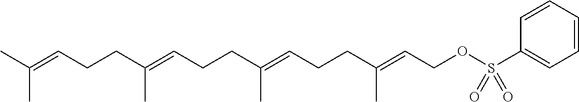 [SMILES: CC(C)=CCC/C(C)=C/CC/C(C)=C/CC/C(C)=C/COS(=O)(=O)c1ccccc1] |
| 4c | 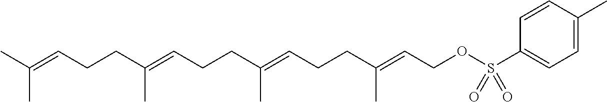 [SMILES: CC(C)=CCC/C(C)=C/CC/C(C)=C/CC/C(C)=C/COS(=O)(=O)c1ccc(C)cc1] |
| 6a | 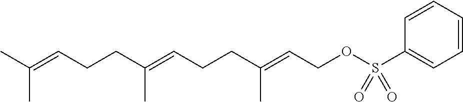 [SMILES: CC(C)=CCC/C(C)=C/CC/C(C)=C/COS(=O)(=O)c1ccccc1] |
| 6b | 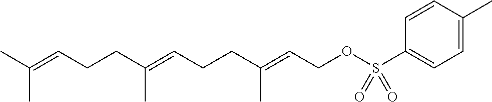 [SMILES: CC(C)=CCC/C(C)=C/CC/C(C)=C/COS(=O)(=O)c1ccc(C)cc1] |
| 7a | 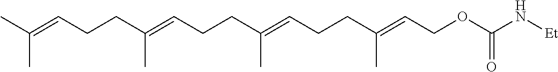 [SMILES: CCNC(=O)OC/C=C(\C)CC/C=C(\C)CC/C=C(\C)CCC=C(C)C] |
| 7b | 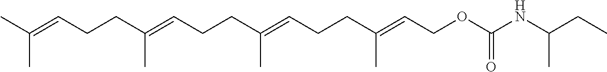 [SMILES: CCC(C)NC(=O)OC/C=C(\C)CC/C=C(\C)CC/C=C(\C)CCC=C(C)C] |
| 7c | 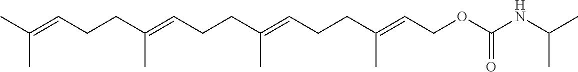 [SMILES: CC(C)=CCC/C(C)=C/CC/C(C)=C/CC/C(C)=C/COC(=O)NC(C)C] |
| 7d | 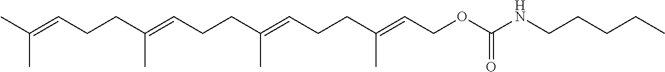 [SMILES: CCCCCNC(=O)OC/C=C(\C)CC/C=C(\C)CC/C=C(\C)CCC=C(C)C] |
| 7e | 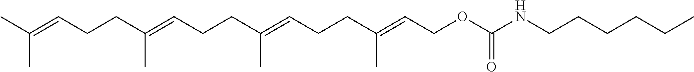 [SMILES: CCCCCCNC(=O)OC/C=C(\C)CC/C=C(\C)CC/C=C(\C)CCC=C(C)C] |
| 7f | 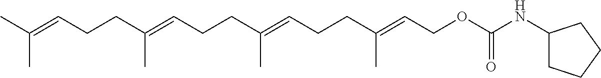 [SMILES: CC(C)=CCC/C(C)=C/CC/C(C)=C/CC/C(C)=C/COC(=O)NC1CCCC1] |
| 7g | 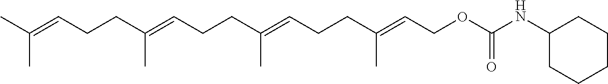 [SMILES: CC(C)=CCC/C(C)=C/CC/C(C)=C/CC/C(C)=C/COC(=O)NC1CCCCC1] |
| 7h | 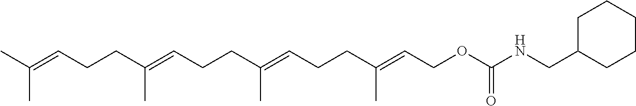 [SMILES: CC(C)=CCC/C(C)=C/CC/C(C)=C/CC/C(C)=C/COC(=O)NCC1CCCCC1] |
| 7i | 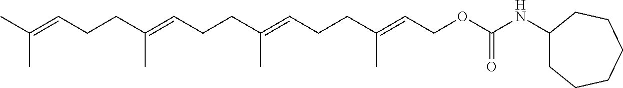 [SMILES: CC(C)=CCC/C(C)=C/CC/C(C)=C/CC/C(C)=C/COC(=O)NC1CCCCCC1] |
| 7j | 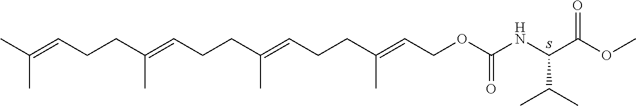 |
| 7k | 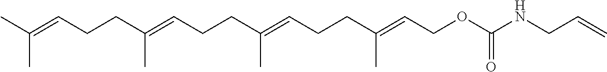 [SMILES: C=CCNC(=O)OC/C=C(\C)CC/C=C(\C)CC/C=C(\C)CCC=C(C)C] |
| 7l | 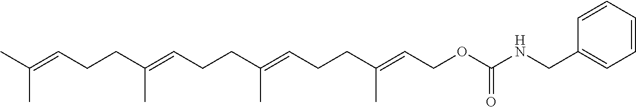 [SMILES: CC(C)=CCC/C(C)=C/CC/C(C)=C/CC/C(C)=C/COC(=O)NCc1ccccc1] |
| 7m | 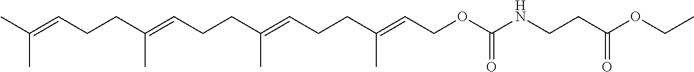 [SMILES: CCOC(=O)CCNC(=O)OC/C=C(\C)CC/C=C(\C)CC/C=C(\C)CCC=C(C)C] |
| 7n | 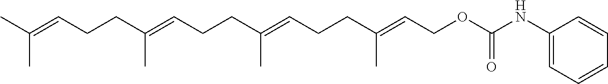 [SMILES: CC(C)=CCC/C(C)=C/CC/C(C)=C/CC/C(C)=C/COC(=O)Nc1ccccc1] |
| 7o | 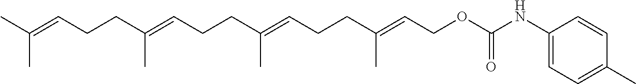 [SMILES: CC(C)=CCC/C(C)=C/CC/C(C)=C/CC/C(C)=C/COC(=O)Nc1ccc(C)cc1] |
| 7p | 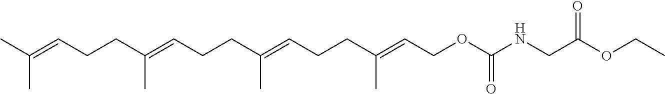 [SMILES: CCOC(=O)CNC(=O)OC/C=C(\C)CC/C=C(\C)CC/C=C(\C)CCC=C(C)C] |
| 7q | 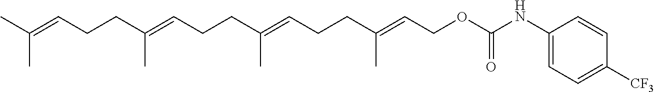 [SMILES: CC(C)=CCC/C(C)=C/CC/C(C)=C/CC/C(C)=C/COC(=O)Nc1ccc(C(F)(F)F)cc1] |
| 7r | 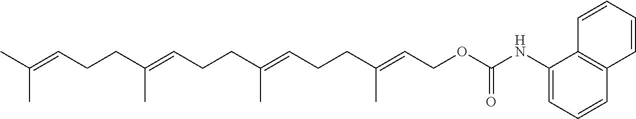 [SMILES: CC(C)=CCC/C(C)=C/CC/C(C)=C/CC/C(C)=C/COC(=O)Nc1cccc2ccccc12] |
| 7s | 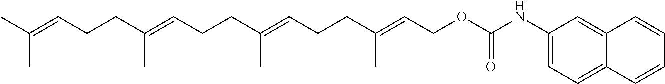 [SMILES: CC(C)=CCC/C(C)=C/CC/C(C)=C/CC/C(C)=C/COC(=O)Nc1ccc2ccccc2c1] |
| 7t | 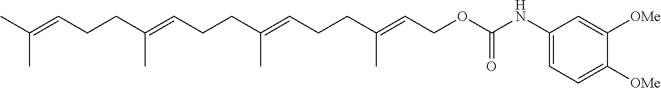 [SMILES: COc1ccc(NC(=O)OC/C=C(\C)CC/C=C(\C)CC/C=C(\C)CCC=C(C)C)cc1OC] |
| 7u | 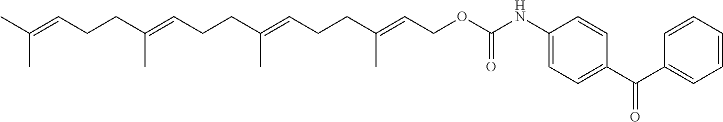 [SMILES: CC(C)=CCC/C(C)=C/CC/C(C)=C/CC/C(C)=C/COC(=O)Nc1ccc(C(=O)c2ccccc2)cc1] |
| 7v | 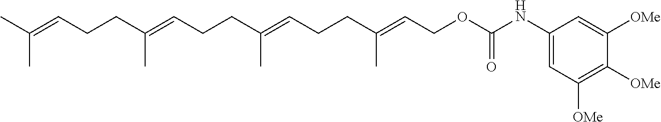 [SMILES: COc1cc(NC(=O)OC/C=C(\C)CC/C=C(\C)CC/C=C(\C)CCC=C(C)C)cc(OC)c1OC] |
| 7w | 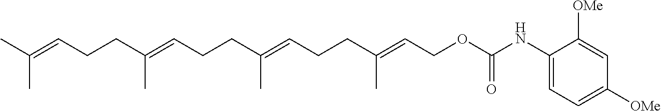 [SMILES: COc1ccc(NC(=O)OC/C=C(\C)CC/C=C(\C)CC/C=C(\C)CCC=C(C)C)c(OC)c1] |
| 7x | 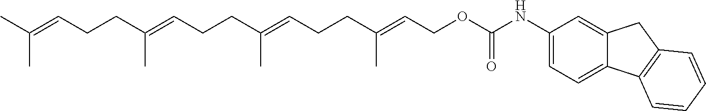 [SMILES: CC(C)=CCC/C(C)=C/CC/C(C)=C/CC/C(C)=C/COC(=O)Nc1ccc2c(c1)Cc1ccccc1-2] |
| 7y | 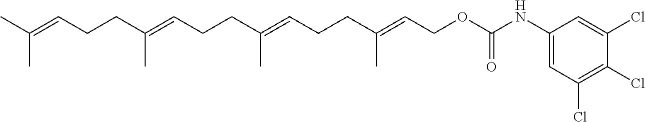 [SMILES: CC(C)=CCC/C(C)=C/CC/C(C)=C/CC/C(C)=C/COC(=O)Nc1cc(Cl)c(Cl)c(Cl)c1] |
| 7z | 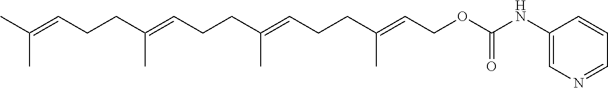 [SMILES: CC(C)=CCC/C(C)=C/CC/C(C)=C/CC/C(C)=C/COC(=O)Nc1cccnc1] |
| 7aa | 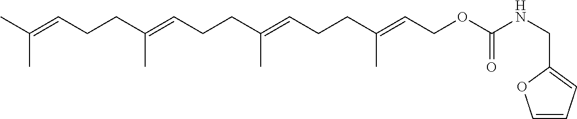 [SMILES: CC(C)=CCC/C(C)=C/CC/C(C)=C/CC/C(C)=C/COC(=O)NCc1ccco1] |
| 8a | 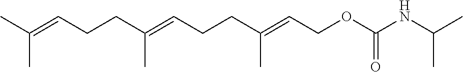 [SMILES: CC(C)=CCC/C(C)=C/CC/C(C)=C/COC(=O)NC(C)C] |
| 8b | 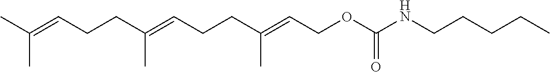 [SMILES: CCCCCNC(=O)OC/C=C(\C)CC/C=C(\C)CCC=C(C)C] |
| 8c | 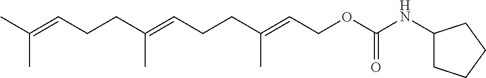 [SMILES: CC(C)=CCC/C(C)=C/CC/C(C)=C/COC(=O)NC1CCCC1] |
| 8d | 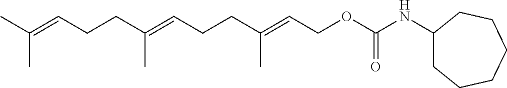 [SMILES: CC(C)=CCC/C(C)=C/CC/C(C)=C/COC(=O)NC1CCCCCC1] |
| 8e | 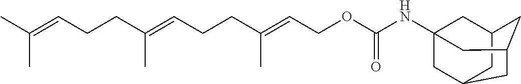 [SMILES: CC(C)=CCC/C(C)=C/CC/C(C)=C/COC(=O)NC12CC3CC(CC(C3)C1)C2] |
| 8f | 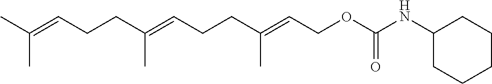 [SMILES: CC(C)=CCC/C(C)=C/CC/C(C)=C/COC(=O)NC1CCCCC1] |
| 8g | 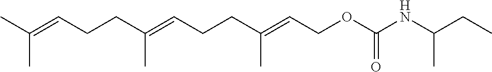 [SMILES: CCC(C)NC(=O)OC/C=C(\C)CC/C=C(\C)CCC=C(C)C] |
| 8h | 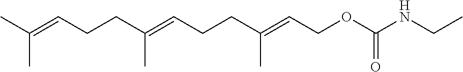 [SMILES: CCNC(=O)OC/C=C(\C)CC/C=C(\C)CCC=C(C)C] |
| 8i | 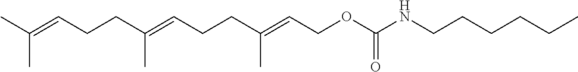 [SMILES: CCCCCCNC(=O)OC/C=C(\C)CC/C=C(\C)CCC=C(C)C] |
| 8j | 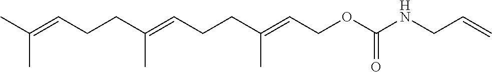 [SMILES: C=CCNC(=O)OC/C=C(\C)CC/C=C(\C)CCC=C(C)C] |
| 8k | 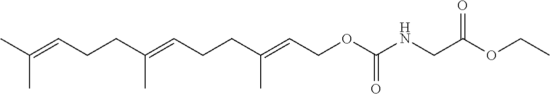 [SMILES: CCOC(=O)CNC(=O)OC/C=C(\C)CC/C=C(\C)CCC=C(C)C] |
| 8l | 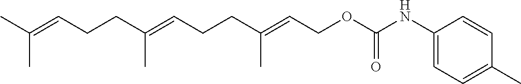 [SMILES: CC(C)=CCC/C(C)=C/CC/C(C)=C/COC(=O)Nc1ccc(C)cc1] |
| 8m | 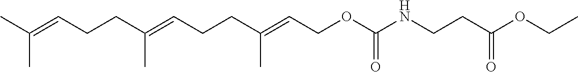 [SMILES: CCOC(=O)CCNC(=O)OC/C=C(\C)CC/C=C(\C)CCC=C(C)C] |
| 8n | 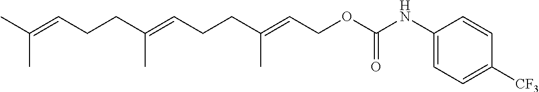 [SMILES: CC(C)=CCC/C(C)=C/CC/C(C)=C/COC(=O)Nc1ccc(C(F)(F)F)cc1] |
| 8o | 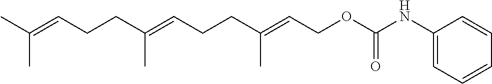 [SMILES: CC(C)=CCC/C(C)=C/CC/C(C)=C/COC(=O)Nc1ccccc1] |
| 9a | 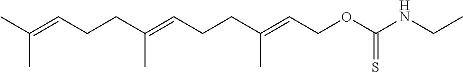 [SMILES: CCNC(=S)OC/C=C(\C)CC/C=C(\C)CCC=C(C)C] |
| 9b | 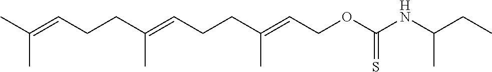 [SMILES: CCC(C)NC(=S)OC/C=C(\C)CC/C=C(\C)CCC=C(C)C] |
| 9c | 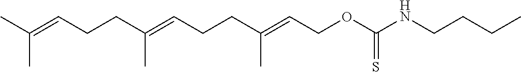 [SMILES: CCCCNC(=S)OC/C=C(\C)CC/C=C(\C)CCC=C(C)C] |
| 9d | 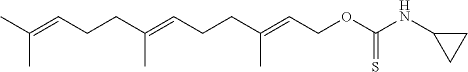 [SMILES: CC(C)=CCC/C(C)=C/CC/C(C)=C/COC(=S)NC1CC1] |
| 9e | 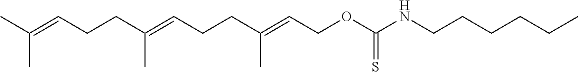 [SMILES: CCCCCCNC(=S)OC/C=C(\C)CC/C=C(\C)CCC=C(C)C] |
| 9f | 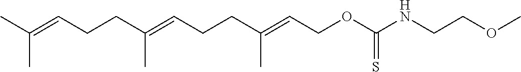 [SMILES: COCCNC(=S)OC/C=C(\C)CC/C=C(\C)CCC=C(C)C] |
| 9g | 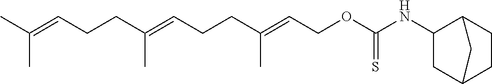 [SMILES: CC(C)=CCC/C(C)=C/CC/C(C)=C/COC(=S)NC1CC2CCC1C2] |
| 9h | 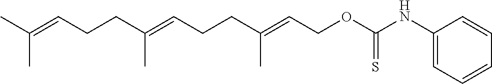 [SMILES: CC(C)=CCC/C(C)=C/CC/C(C)=C/COC(=S)Nc1ccccc1] |
| 9i | 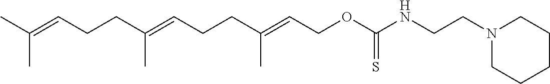 [SMILES: CC(C)=CCC/C(C)=C/CC/C(C)=C/COC(=S)NCCN1CCCCC1] |
| 9j | 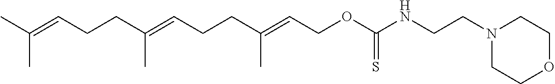 [SMILES: CC(C)=CCC/C(C)=C/CC/C(C)=C/COC(=S)NCCN1CCOCC1] |
| 9k | 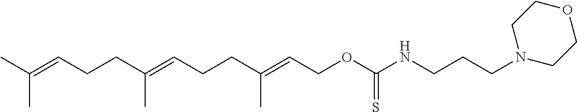 [SMILES: CC(C)=CCC/C(C)=C/CC/C(C)=C/COC(=S)NCCCN1CCOCC1] |
| 10a | 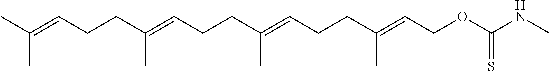 [SMILES: CNC(=S)OC/C=C(\C)CC/C=C(\C)CC/C=C(\C)CCC=C(C)C] |
| 10b | 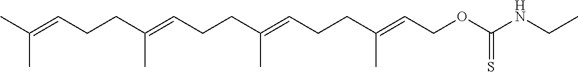 [SMILES: CCNC(=S)OC/C=C(\C)CC/C=C(\C)CC/C=C(\C)CCC=C(C)C] |
| 10c | 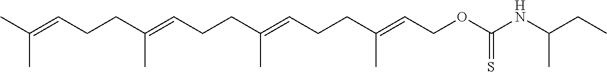 [SMILES: CCC(C)NC(=S)OC/C=C(\C)CC/C=C(\C)CC/C=C(\C)CCC=C(C)C] |
| 10d | 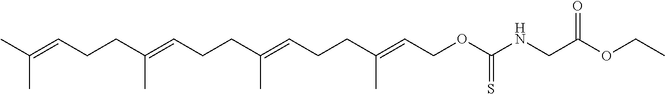 [SMILES: CCOC(=O)CNC(=S)OC/C=C(\C)CC/C=C(\C)CC/C=C(\C)CCC=C(C)C] |
| 10e | 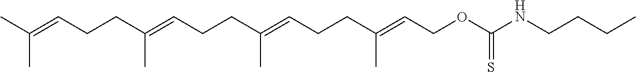 [SMILES: CCCCNC(=S)OC/C=C(\C)CC/C=C(\C)CC/C=C(\C)CCC=C(C)C] |
| 10f | 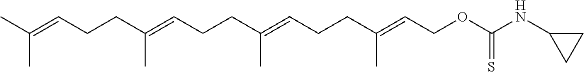 [SMILES: CC(C)=CCC/C(C)=C/CC/C(C)=C/CC/C(C)=C/COC(=S)NC1CC1] |
| 10g | 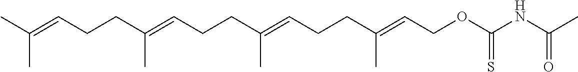 [SMILES: CC(=O)NC(=S)OC/C=C(\C)CC/C=C(\C)CC/C=C(\C)CCC=C(C)C] |
| 10h | 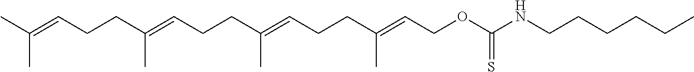 [SMILES: CCCCCCNC(=S)OC/C=C(\C)CC/C=C(\C)CC/C=C(\C)CCC=C(C)C] |
| 10i | 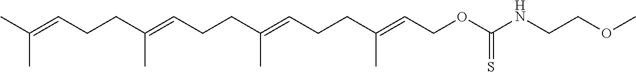 [SMILES: COCCNC(=S)OC/C=C(\C)CC/C=C(\C)CC/C=C(\C)CCC=C(C)C] |
| 10j | 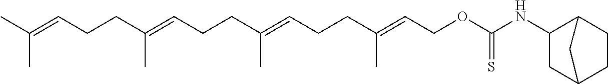 [SMILES: CC(C)=CCC/C(C)=C/CC/C(C)=C/CC/C(C)=C/COC(=S)NC1CC2CCC1C2] |
| 10k | 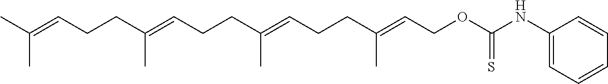 [SMILES: CC(C)=CCC/C(C)=C/CC/C(C)=C/CC/C(C)=C/COC(=S)Nc1ccccc1] |
| 10l | 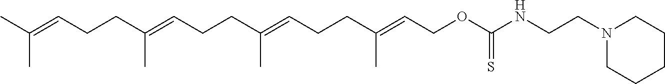 [SMILES: CC(C)=CCC/C(C)=C/CC/C(C)=C/CC/C(C)=C/COC(=S)NCCN1CCCCC1] |
| 10m | 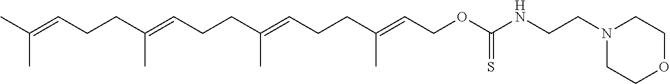 [SMILES: CC(C)=CCC/C(C)=C/CC/C(C)=C/CC/C(C)=C/COC(=S)NCCN1CCOCC1] |
| 12 | 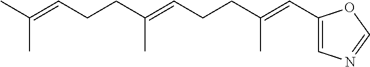 [SMILES: CC(C)=CCC/C(C)=C/CC/C(C)=C/c1cnco1] |
| 14 | 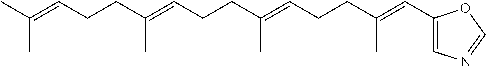 [SMILES: CC(C)=CCC/C(C)=C/CC/C(C)=C/CC/C(C)=C/c1cnco1] |
| 15 | 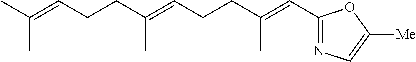 [SMILES: CC(C)=CCC/C(C)=C/CC/C(C)=C/c1ncc(C)o1] |
| 16 | 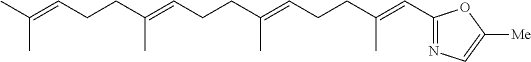 [SMILES: CC(C)=CCC/C(C)=C/CC/C(C)=C/CC/C(C)=C/c1ncc(C)o1] |
| 17a | 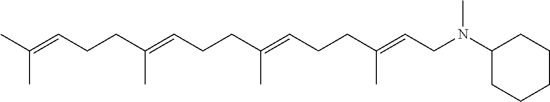 [SMILES: CC(C)=CCC/C(C)=C/CC/C(C)=C/CC/C(C)=C/CN(C)C1CCCCC1] |
| 17b | 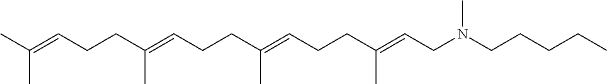 [SMILES: CCCCCN(C)C/C=C(\C)CC/C=C(\C)CC/C=C(\C)CCC=C(C)C] |
| 17c | 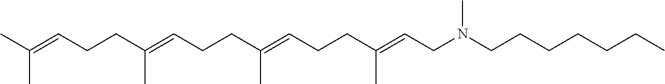 [SMILES: CCCCCCCN(C)C/C=C(\C)CC/C=C(\C)CC/C=C(\C)CCC=C(C)C] |
| 17d | 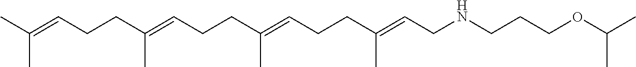 [SMILES: CC(C)=CCC/C(C)=C/CC/C(C)=C/CC/C(C)=C/CNCCCOC(C)C] |
| 17e | 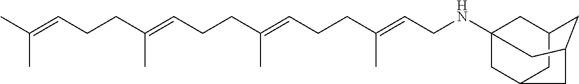 [SMILES: CC(C)=CCC/C(C)=C/CC/C(C)=C/CC/C(C)=C/CNC12CC3CC(CC(C3)C1)C2] |
| 19 | 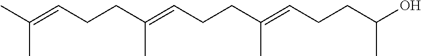 [SMILES: CC(C)=CCC/C(C)=C/CC/C(C)=C/CCC(C)O] |
| 20a | 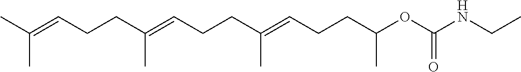 [SMILES: CCNC(=O)OC(C)CC/C=C(\C)CC/C=C(\C)CCC=C(C)C] |
| 20b | 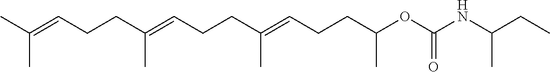 [SMILES: CCC(C)NC(=O)OC(C)CC/C=C(\C)CC/C=C(\C)CCC=C(C)C] |
| 20c | 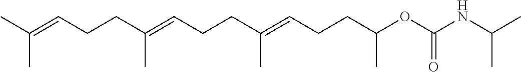 [SMILES: CC(C)=CCC/C(C)=C/CC/C(C)=C/CCC(C)OC(=O)NC(C)C] |
| 20d | 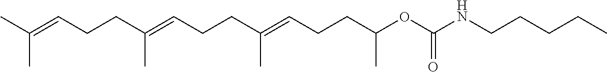 [SMILES: CCCCCNC(=O)OC(C)CC/C=C(\C)CC/C=C(\C)CCC=C(C)C] |
| 20e | 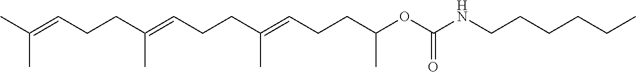 [SMILES: CCCCCCNC(=O)OC(C)CC/C=C(\C)CC/C=C(\C)CCC=C(C)C] |
| 20f | 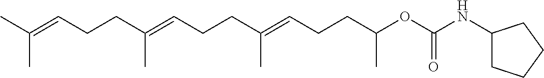 [SMILES: CC(C)=CCC/C(C)=C/CC/C(C)=C/CCC(C)OC(=O)NC1CCCC1] |
| 20g | 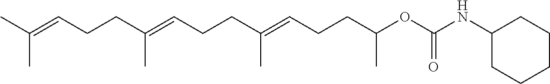 [SMILES: CC(C)=CCC/C(C)=C/CC/C(C)=C/CCC(C)OC(=O)NC1CCCCC1] |
| 20h | 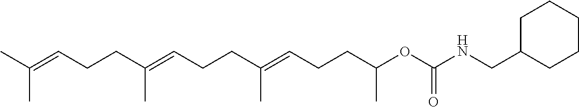 [SMILES: CC(C)=CCC/C(C)=C/CC/C(C)=C/CCC(C)OC(=O)NCC1CCCCC1] |
| 20i | 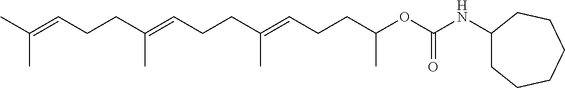 [SMILES: CC(C)=CCC/C(C)=C/CC/C(C)=C/CCC(C)OC(=O)NC1CCCCCC1] |
| 20j | 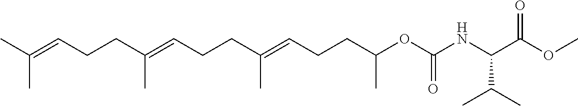 [SMILES: COC(=O)[C@@H](NC(=O)OC(C)CC/C=C(\C)CC/C=C(\C)CCC=C(C)C)C(C)C] |
| 22 | 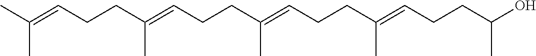 [SMILES: CC(C)=CCC/C(C)=C/CC/C(C)=C/CC/C(C)=C/CCC(C)O] |
| 23a | 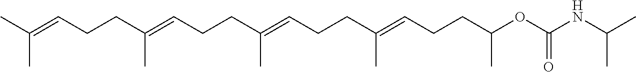 [SMILES: CC(C)=CCC/C(C)=C/CC/C(C)=C/CC/C(C)=C/CCC(C)OC(=O)NC(C)C] |
| 23b | 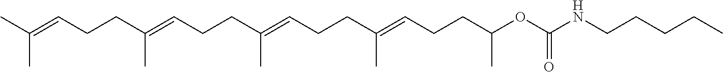 [SMILES: CCCCCNC(=O)OC(C)CC/C=C(\C)CC/C=C(\C)CC/C=C(\C)CCC=C(C)C] |
| 23c | 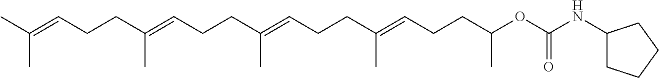 [SMILES: CC(C)=CCC/C(C)=C/CC/C(C)=C/CC/C(C)=C/CCC(C)OC(=O)NC1CCCC1] |
| 23d | 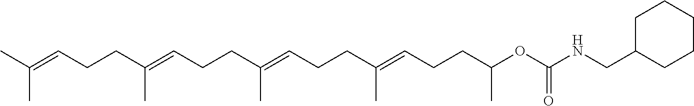 [SMILES: CC(C)=CCC/C(C)=C/CC/C(C)=C/CC/C(C)=C/CCC(C)OC(=O)NCC1CCCCC1] |
| 23e | 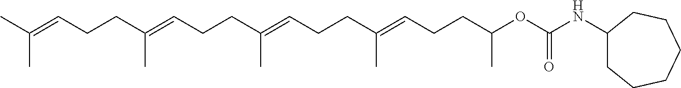 [SMILES: CC(C)=CCC/C(C)=C/CC/C(C)=C/CC/C(C)=C/CCC(C)OC(=O)NC1CCCCCC1] |
| 23f | 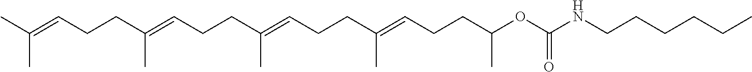 [SMILES: CCCCCCNC(=O)OC(C)CC/C=C(\C)CC/C=C(\C)CC/C=C(\C)CCC=C(C)C] |
| 23g | 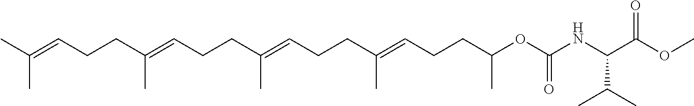 [SMILES: COC(=O)[C@@H](NC(=O)OC(C)CC/C=C(\C)CC/C=C(\C)CC/C=C(\C)CCC=C(C)C)C(C)C] |
| 24 | 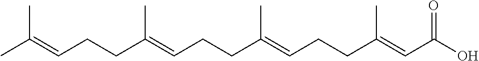 [SMILES: CC(C)=CCC/C(C)=C/CC/C(C)=C/CC/C(C)=C/C(=O)O] |
| 25 | 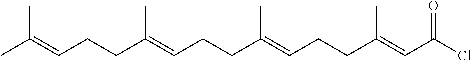 [SMILES: CC(C)=CCC/C(C)=C/CC/C(C)=C/CC/C(C)=C/C(=O)Cl] |
| 27a | 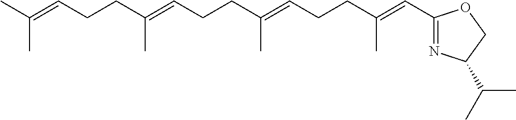 [SMILES: CC(C)=CCC/C(C)=C/CC/C(C)=C/CC/C(C)=C/C1=N[C@@H](C(C)C)CO1] |
| 27b | 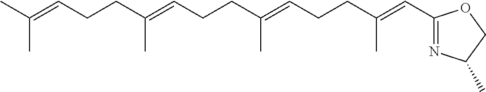 [SMILES: CC(C)=CCC/C(C)=C/CC/C(C)=C/CC/C(C)=C/C1=N[C@@H](C)CO1] |
| 27c | 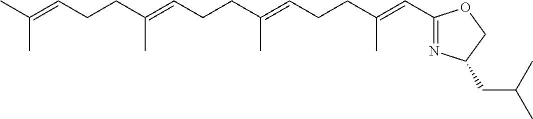 [SMILES: CC(C)=CCC/C(C)=C/CC/C(C)=C/CC/C(C)=C/C1=N[C@@H](CC(C)C)CO1] |
| 27d | 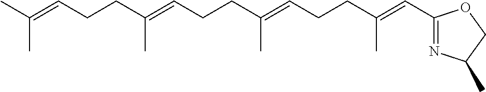 [SMILES: CC(C)=CCC/C(C)=C/CC/C(C)=C/CC/C(C)=C/C1=N[C@H](C)CO1] |
| 27e | 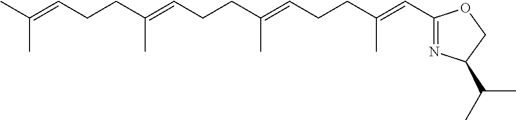 [SMILES: CC(C)=CCC/C(C)=C/CC/C(C)=C/CC/C(C)=C/C1=N[C@H](C(C)C)CO1] |
| 27f | 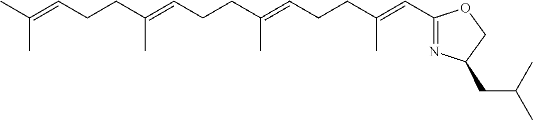 [SMILES: CC(C)=CCC/C(C)=C/CC/C(C)=C/CC/C(C)=C/C1=N[C@H](CC(C)C)CO1] |
| 27g | 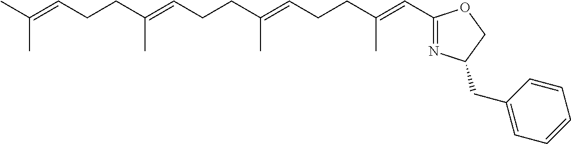 [SMILES: CC(C)=CCC/C(C)=C/CC/C(C)=C/CC/C(C)=C/C1=N[C@@H](Cc2ccccc2)CO1] |
| 29a | 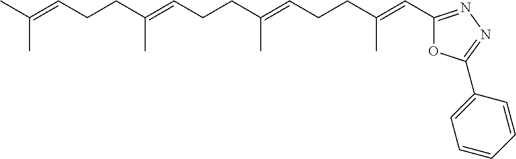 [SMILES: CC(C)=CCC/C(C)=C/CC/C(C)=C/CC/C(C)=C/c1nnc(-c2ccccc2)o1] |
| 29b | 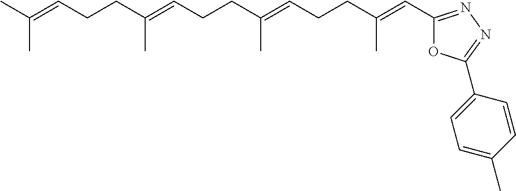 [SMILES: CC(C)=CCC/C(C)=C/CC/C(C)=C/CC/C(C)=C/c1nnc(-c2ccc(C)cc2)o1] |
| 29c | 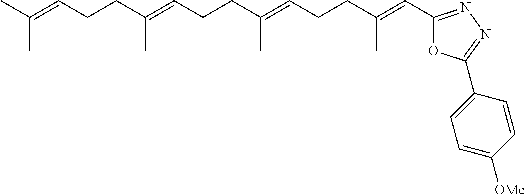 [SMILES: COc1ccc(-c2nnc(/C=C(\C)CC/C=C(\C)CC/C=C(\C)CCC=C(C)C)o2)cc1] |
| 29d | 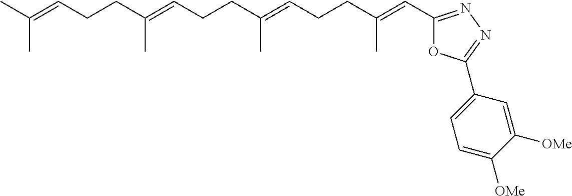 [SMILES: COc1ccc(-c2nnc(/C=C(\C)CC/C=C(\C)CC/C=C(\C)CCC=C(C)C)o2)cc1OC] |
| 29e | 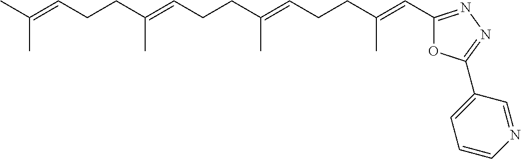 [SMILES: CC(C)=CCC/C(C)=C/CC/C(C)=C/CC/C(C)=C/c1nnc(-c2cccnc2)o1] |
| 29f | 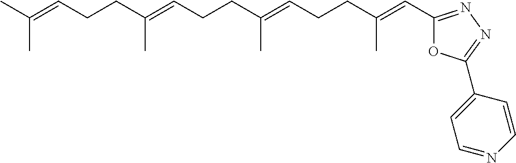 [SMILES: CC(C)=CCC/C(C)=C/CC/C(C)=C/CC/C(C)=C/c1nnc(-c2ccncc2)o1] |
| 31 | 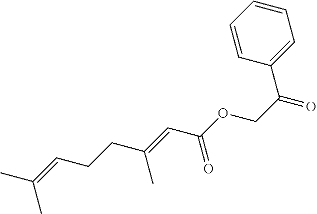 [SMILES: CC(C)=CCC/C(C)=C/C(=O)OCC(=O)c1ccccc1] |
| 32 | 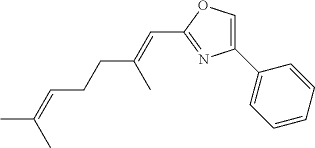 [SMILES: CC(C)=CCC/C(C)=C/c1nc(-c2ccccc2)co1] |
| 35a | 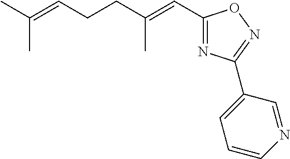 [SMILES: CC(C)=CCC/C(C)=C/c1nc(-c2cccnc2)no1] |
| 35b | 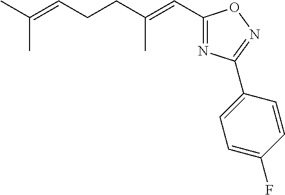 [SMILES: CC(C)=CCC/C(C)=C/c1nc(-c2ccc(F)cc2)no1] |
| 35c | 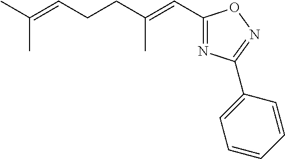 [SMILES: CC(C)=CCC/C(C)=C/c1nc(-c2ccccc2)no1] |
| 35d | 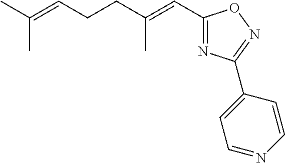 [SMILES: CC(C)=CCC/C(C)=C/c1nc(-c2ccncc2)no1] |
| 37a | 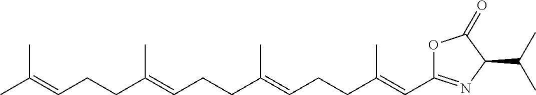 [SMILES: CC(C)=CCC/C(C)=C/CC/C(C)=C/CC/C(C)=C/C1=N[C@H](C(C)C)C(=O)O1] |
| 37b | 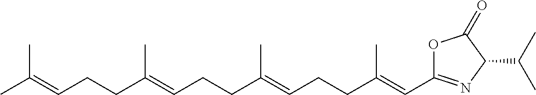 [SMILES: CC(C)=CCC/C(C)=C/CC/C(C)=C/CC/C(C)=C/C1=N[C@@H](C(C)C)C(=O)O1] |
| 37c | 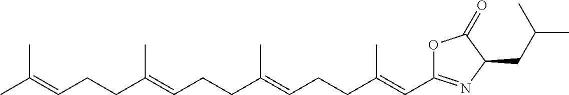 [SMILES: CC(C)=CCC/C(C)=C/CC/C(C)=C/CC/C(C)=C/C1=N[C@H](CC(C)C)C(=O)O1] |
| 37d | 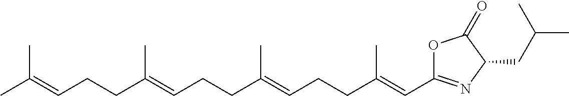 [SMILES: CC(C)=CCC/C(C)=C/CC/C(C)=C/CC/C(C)=C/C1=N[C@@H](CC(C)C)C(=O)O1] |
| 38a | 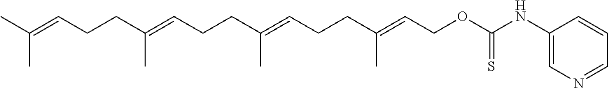 [SMILES: CC(C)=CCC/C(C)=C/CC/C(C)=C/CC/C(C)=C/COC(=S)Nc1cccnc1] |
| 38b | 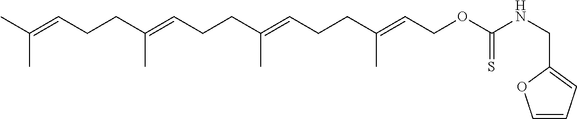 [SMILES: CC(C)=CCC/C(C)=C/CC/C(C)=C/CC/C(C)=C/COC(=S)NCc1ccco1] |
| 39 | 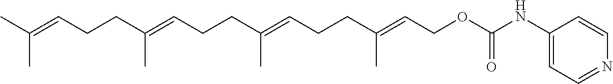 [SMILES: CC(C)=CCC/C(C)=C/CC/C(C)=C/CC/C(C)=C/COC(=O)Nc1ccncc1] |
| 40a | 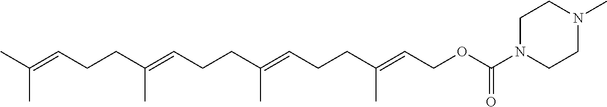 [SMILES: CC(C)=CCC/C(C)=C/CC/C(C)=C/CC/C(C)=C/COC(=O)N1CCN(C)CC1] |
| 40b | 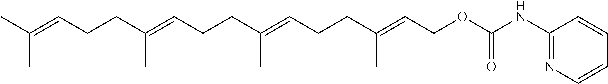 [SMILES: CC(C)=CCC/C(C)=C/CC/C(C)=C/CC/C(C)=C/COC(=O)Nc1ccccn1] |
| 41 | 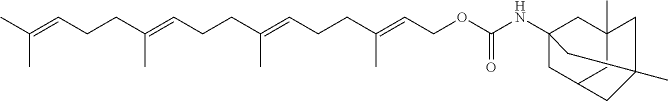 [SMILES: CC(C)=CCC/C(C)=C/CC/C(C)=C/CC/C(C)=C/COC(=O)NC12CC3CC(C)(CC(C)(C3)C1)C2] |
| 42 | 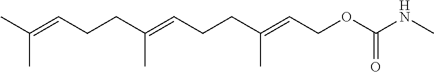 [SMILES: CNC(=O)OC/C=C(\C)CC/C=C(\C)CCC=C(C)C] |
| 43 | 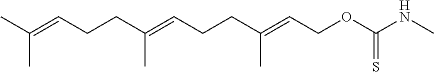 [SMILES: CNC(=S)OC/C=C(\C)CC/C=C(\C)CCC=C(C)C] |

In another embodiment, examples of compounds provided and/or utilized by this invention include certain compounds tabulated below.

TABLE 2

| Compound | |
|---|---|
| ID | Chemical Structure |
| 51 | 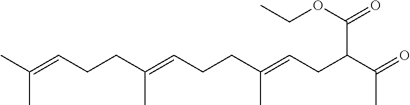 [SMILES: CCOC(=O)C(C/C=C(\C)CC/C=C(\C)CCC=C(C)C)C(C)=O] |
| 52 | 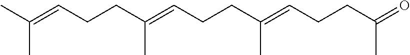 [SMILES: CC(=O)CC/C=C(\C)CC/C=C(\C)CCC=C(C)C] |
| 54 | 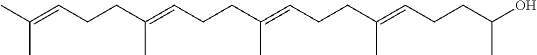 [SMILES: CC(C)=CCC/C(C)=C/CC/C(C)=C/CC/C(C)=C/CCC(C)O] |
| 55 | 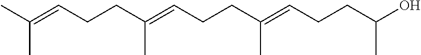 [SMILES: CC(C)=CCC/C(C)=C/CC/C(C)=C/CCC(C)O] |
| 56 | 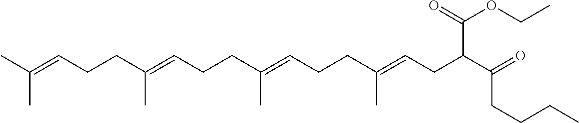 [SMILES: CCCCC(=O)C(C/C=C(\C)CC/C=C(\C)CC/C=C(\C)CCC=C(C)C)C(=O)OCC] |
| 57 | 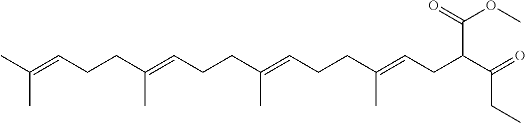 [SMILES: CCC(=O)C(C/C=C(\C)CC/C=C(\C)CC/C=C(\C)CCC=C(C)C)C(=O)OC] |
| 58 | 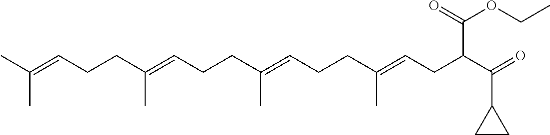 [SMILES: CCOC(=O)C(C/C=C(\C)CC/C=C(\C)CC/C=C(\C)CCC=C(C)C)C(=O)C1CC1] |
| 59 | 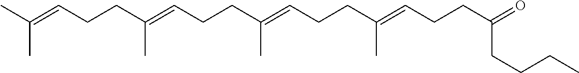 [SMILES: CCCCC(=O)CC/C=C(\C)CC/C=C(\C)CC/C=C(\C)CCC=C(C)C] |
| 60 | 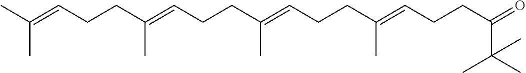 [SMILES: CC(C)=CCC/C(C)=C/CC/C(C)=C/CC/C(C)=C/CCC(=O)C(C)(C)C] |
| 61 | 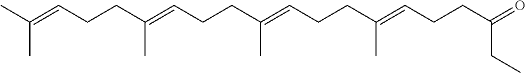 [SMILES: CCC(=O)CC/C=C(\C)CC/C=C(\C)CC/C=C(\C)CCC=C(C)C] |
| 62 | 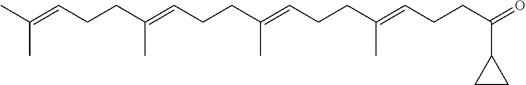 [SMILES: CC(C)=CCC/C(C)=C/CC/C(C)=C/CC/C(C)=C/CCC(=O)C1CC1] |
| 63 | 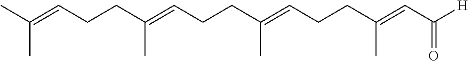 [SMILES: CC(C)=CCC/C(C)=C/CC/C(C)=C/CC/C(C)=C/C=O] |
| 64 | 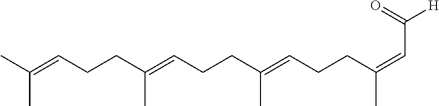 [SMILES: CC(C)=CCC/C(C)=C/CC/C(C)=C/CC/C(C)=C\C=O] |
| 65 | 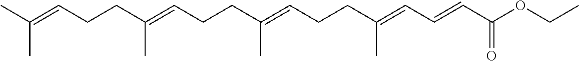 [SMILES: CCOC(=O)/C=C/C=C(\C)CC/C=C(\C)CC/C=C(\C)CCC=C(C)C] |
| 66 | 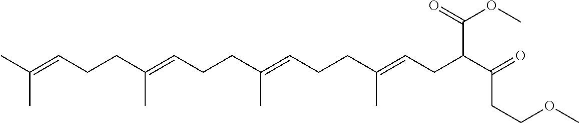 [SMILES: COCCC(=O)C(C/C=C(\C)CC/C=C(\C)CC/C=C(\C)CCC=C(C)C)C(=O)OC] |
| 67 | 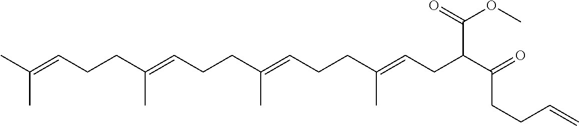 [SMILES: C=CCCC(=O)C(C/C=C(\C)CC/C=C(\C)CC/C=C(\C)CCC=C(C)C)C(=O)OC] |
| 68 | 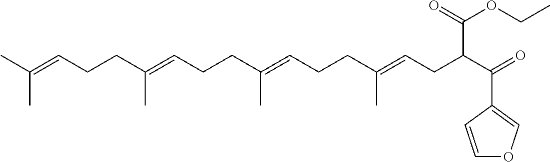 [SMILES: CCOC(=O)C(C/C=C(\C)CC/C=C(\C)CC/C=C(\C)CCC=C(C)C)C(=O)c1ccoc1] |
| 69 | 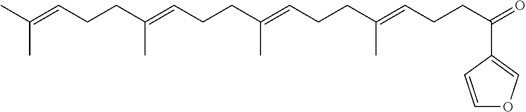 [SMILES: CC(C)=CCC/C(C)=C/CC/C(C)=C/CC/C(C)=C/CCC(=O)c1ccoc1] |
| 70 | 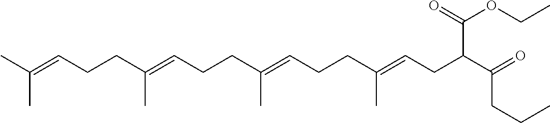 [SMILES: CCCC(=O)C(C/C=C(\C)CC/C=C(\C)CC/C=C(\C)CCC=C(C)C)C(=O)OCC] |
| 71 | 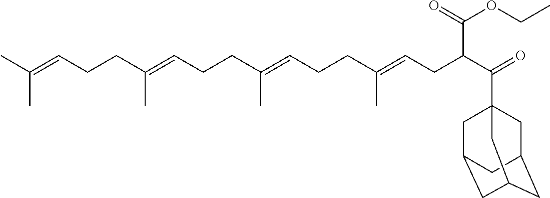 [SMILES: CCOC(=O)C(C/C=C(\C)CC/C=C(\C)CC/C=C(\C)CCC=C(C)C)C(=O)C12CC3CC(CC(C3)C1)C2] |
| 72 | 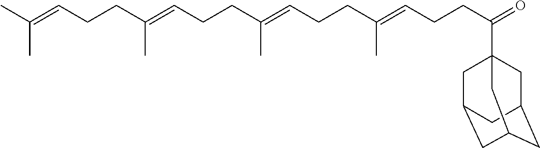 [SMILES: CC(C)=CCC/C(C)=C/CC/C(C)=C/CC/C(C)=C/CCC(=O)C12CC3CC(CC(C3)C1)C2] |
| 73 | 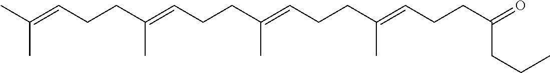 [SMILES: CCCC(=O)CC/C=C(\C)CC/C=C(\C)CC/C=C(\C)CCC=C(C)C] |
| 74 | 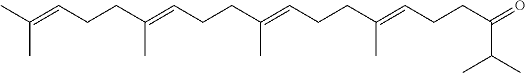 [SMILES: CC(C)=CCC/C(C)=C/CC/C(C)=C/CC/C(C)=C/CCC(=O)C(C)C] |
| 75 | 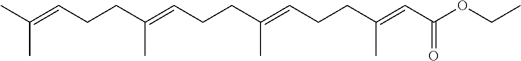 [SMILES: CCOC(=O)/C=C(\C)CC/C=C(\C)CC/C=C(\C)CCC=C(C)C] |
| 76 | 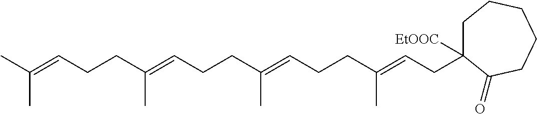 [SMILES: CCOC(=O)C1(C/C=C(\C)CC/C=C(\C)CC/C=C(\C)CCC=C(C)C)CCCCCC1=O] |
| 77 | 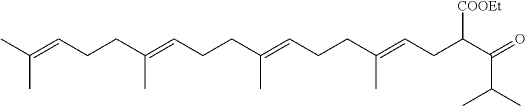 [SMILES: CCOC(=O)C(C/C=C(\C)CC/C=C(\C)CC/C=C(\C)CCC=C(C)C)C(=O)C(C)C] |
| 78 | 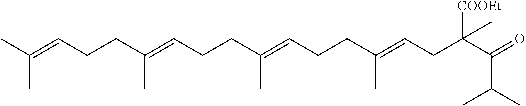 [SMILES: CCOC(=O)C(C)(C/C=C(\C)CC/C=C(\C)CC/C=C(\C)CCC=C(C)C)C(=O)C(C)C] |
| 6979 | 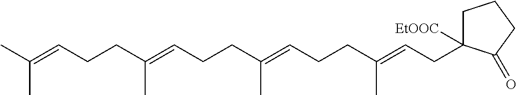 [SMILES: CCOC(=O)C1(C/C=C(\C)CC/C=C(\C)CC/C=C(\C)CCC=C(C)C)CCCC1=O] |
| 80 | 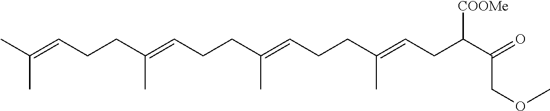 [SMILES: COCC(=O)C(C/C=C(\C)CC/C=C(\C)CC/C=C(\C)CCC=C(C)C)C(=O)OC] |
| 81 | 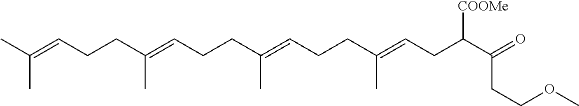 [SMILES: COCCC(=O)C(C/C=C(\C)CC/C=C(\C)CC/C=C(\C)CCC=C(C)C)C(=O)OC] |
| 82 | 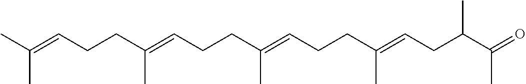 [SMILES: CC(=O)C(C)C/C=C(\C)CC/C=C(\C)CC/C=C(\C)CCC=C(C)C] |
| 83 | 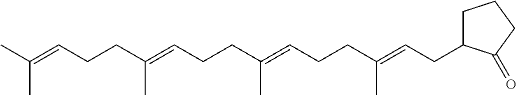 [SMILES: CC(C)=CCC/C(C)=C/CC/C(C)=C/CC/C(C)=C/CC1CCCC1=O] |
| 84 | 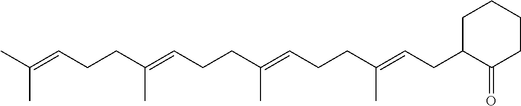 [SMILES: CC(C)=CCC/C(C)=C/CC/C(C)=C/CC/C(C)=C/CC1CCCCC1=O] |
| 85 | 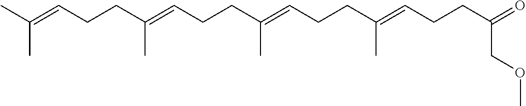 [SMILES: COCC(=O)CC/C=C(\C)CC/C=C(\C)CC/C=C(\C)CCC=C(C)C] |
| 86 | 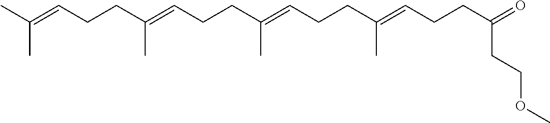 [SMILES: COCCC(=O)CC/C=C(\C)CC/C=C(\C)CC/C=C(\C)CCC=C(C)C] |
| 87 | 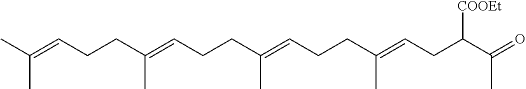 [SMILES: CCOC(=O)C(C/C=C(\C)CC/C=C(\C)CC/C=C(\C)CCC=C(C)C)C(C)=O] |
| 88 | 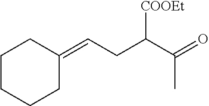 [SMILES: CCOC(=O)C(CC=C1CCCCC1)C(C)=O] |
| 89 | 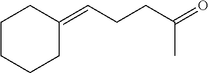 |
| 90 | 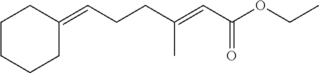 [SMILES: CCOC(=O)/C=C(\C)CCC=C1CCCCC1] |
| 91 | 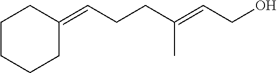 |
| 92 | 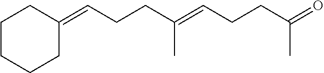 [SMILES: CC(=O)CC/C=C(\C)CCC=C1CCCCC1] |
| 93 | 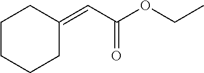 |
| 94 | 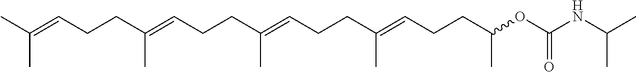 [SMILES: CC(C)=CCC/C(C)=C/CC/C(C)=C/CC/C(C)=C/CCC(C)OC(=O)NC(C)C] |
| 95 | 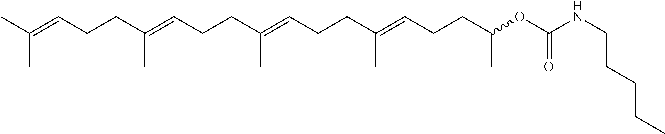 [SMILES: CCCCCNC(=O)OC(C)CC/C=C(\C)CC/C=C(\C)CC/C=C(\C)CCC=C(C)C] |
| 96 | 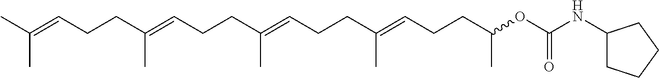 [SMILES: CC(C)=CCC/C(C)=C/CC/C(C)=C/CC/C(C)=C/CCC(C)OC(=O)NC1CCCC1] |
| 97 | 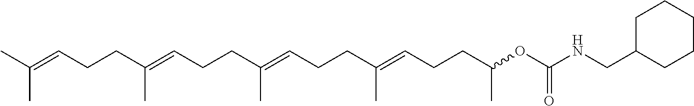 [SMILES: CC(C)=CCC/C(C)=C/CC/C(C)=C/CC/C(C)=C/CCC(C)OC(=O)NCC1CCCCC1] |
| 98 | 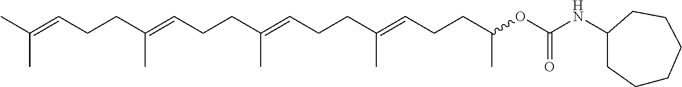 [SMILES: CC(C)=CCC/C(C)=C/CC/C(C)=C/CC/C(C)=C/CCC(C)OC(=O)NC1CCCCCC1] |
| 99 | 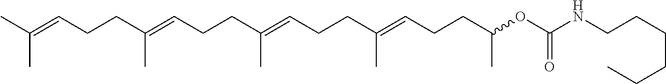 [SMILES: CCCCCCNC(=O)OC(C)CC/C=C(\C)CC/C=C(\C)CC/C=C(\C)CCC=C(C)C] |
| 100 | 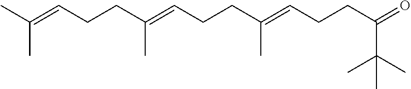 [SMILES: CC(C)=CCC/C(C)=C/CC/C(C)=C/CCC(=O)C(C)(C)C] |
| 101 | 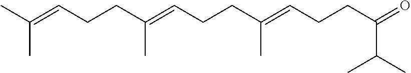 [SMILES: CC(C)=CCC/C(C)=C/CC/C(C)=C/CCC(=O)C(C)C] |
| 102 | 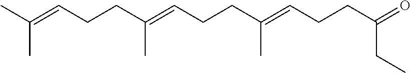 [SMILES: CCC(=O)CC/C=C(\C)CC/C=C(\C)CCC=C(C)C] |
| 103 | 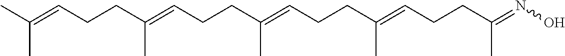 [SMILES: CC(C)=CCC/C(C)=C/CC/C(C)=C/CC/C(C)=C/CCC(C)=NO] |
| 104 | 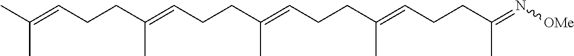 [SMILES: CON=C(C)CC/C=C(\C)CC/C=C(\C)CC/C=C(\C)CCC=C(C)C] |
| 105 | 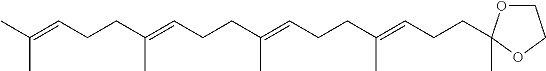 [SMILES: CC(C)=CCC/C(C)=C/CC/C(C)=C/CC/C(C)=C/CCC1(C)OCCO1] |
| 106 | 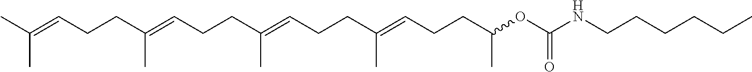 [SMILES: CCCCCCNC(=O)OC(C)CC/C=C(\C)CC/C=C(\C)CC/C=C(\C)CCC=C(C)C] |
| 107 | 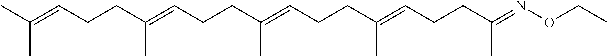 [SMILES: CCO/N=C(\C)CC/C=C(\C)CC/C=C(\C)CC/C=C(\C)CCC=C(C)C] |
| 108 | 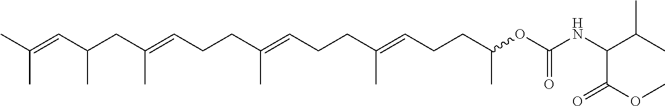 [SMILES: COC(=O)C(NC(=O)OC(C)CC/C=C(\C)CC/C=C(\C)CC/C=C(\C)CC(C)C=C(C)C)C(C)C] |
| 109 | 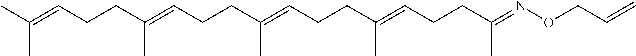 [SMILES: C=CCO/N=C(\C)CC/C=C(\C)CC/C=C(\C)CC/C=C(\C)CCC=C(C)C] |
| 110 | 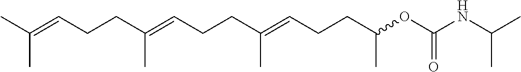 [SMILES: CC(C)=CCC/C(C)=C/CC/C(C)=C/CCC(C)OC(=O)NC(C)C] |
| 111 | 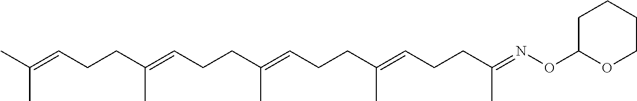 [SMILES: CC(C)=CCC/C(C)=C/CC/C(C)=C/CC/C(C)=C/CC/C(C)=N/OC1CCCCO1] |
| 112 | 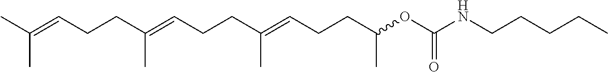 [SMILES: CCCCCNC(=O)OC(C)CC/C=C(\C)CC/C=C(\C)CCC=C(C)C] |
| 113 | 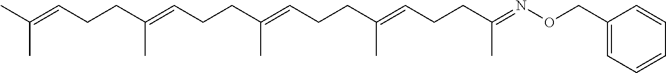 [SMILES: CC(C)=CCC/C(C)=C/CC/C(C)=C/CC/C(C)=C/CC/C(C)=N/OCc1ccccc1] |
| 114 | 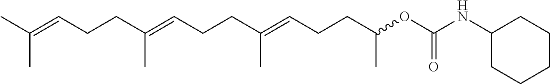 [SMILES: CC(C)=CCC/C(C)=C/CC/C(C)=C/CCC(C)OC(=O)NC1CCCCC1] |
| 115 | 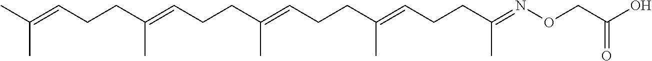 [SMILES: CC(C)=CCC/C(C)=C/CC/C(C)=C/CC/C(C)=C/CC/C(C)=N/OCC(=O)O] |
| 116 | 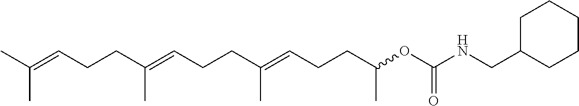 [SMILES: CC(C)=CCC/C(C)=C/CC/C(C)=C/CCC(C)OC(=O)NCC1CCCCC1] |
| 117 | 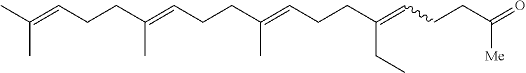 [SMILES: CCC(=CCCC(C)=O)CC/C=C(\C)CC/C=C(\C)CCC=C(C)C] |
| 118 | 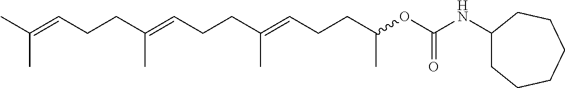 [SMILES: CC(C)=CCC/C(C)=C/CC/C(C)=C/CCC(C)OC(=O)NC1CCCCCC1] |
| 119 | 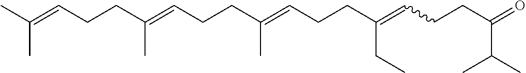 [SMILES: CCC(=CCCC(=O)C(C)C)CC/C=C(\C)CC/C=C(\C)CCC=C(C)C] |
| 120 | 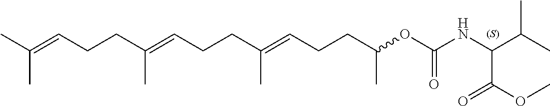 |
| 121 | 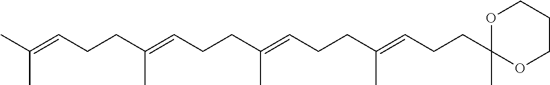 [SMILES: CC(C)=CCC/C(C)=C/CC/C(C)=C/CC/C(C)=C/CCC1(C)OCCCO1] |
| 122 | 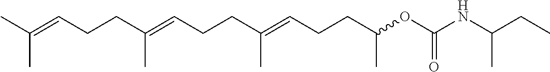 [SMILES: CCC(C)NC(=O)OC(C)CC/C=C(\C)CC/C=C(\C)CCC=C(C)C] |
| 123 | 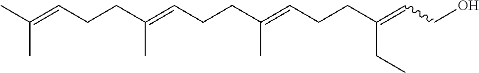 [SMILES: CCC(=CCO)CC/C=C(\C)CC/C=C(\C)CCC=C(C)C] |
| 124 | 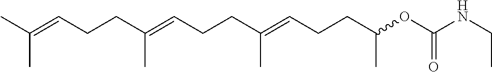 [SMILES: CCNC(=O)OC(C)CC/C=C(\C)CC/C=C(\C)CCC=C(C)C] |
| 125 | 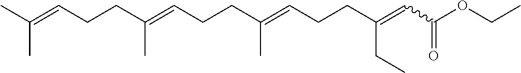 [SMILES: CCOC(=O)C=C(CC)CC/C=C(\C)CC/C=C(\C)CCC=C(C)C] |
| 126 | 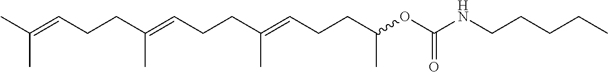 [SMILES: CCCCCNC(=O)OC(C)CC/C=C(\C)CC/C=C(\C)CCC=C(C)C] |
| 127 | 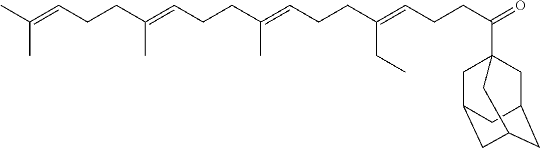 [SMILES: CC/C(=C\CCC(=O)C12CC3CC(CC(C3)C1)C2)CC/C=C(\C)CC/C=C(\C)CCC=C(C)C] |
| 128 | 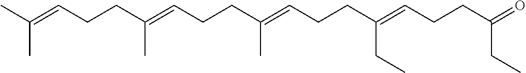 [SMILES: CCC(=O)CC/C=C(\CC)CC/C=C(\C)CC/C=C(\C)CCC=C(C)C] |
| 129 | 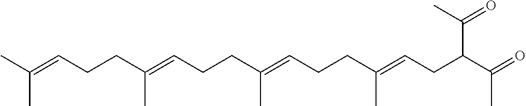 [SMILES: CC(=O)C(C/C=C(\C)CC/C=C(\C)CC/C=C(\C)CCC=C(C)C)C(C)=O] |
| 130 | 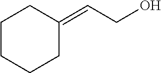 |
| 131 | 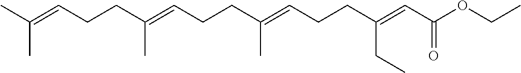 [SMILES: CCOC(=O)/C=C(\CC)CC/C=C(\C)CC/C=C(\C)CCC=C(C)C] |
| 132 | 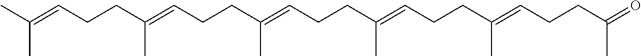 [SMILES: CC(=O)CC/C=C(\C)CC/C=C(\C)CC/C=C(\C)CC/C=C(\C)CCC=C(C)C] |
| 133 | 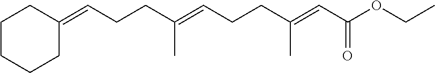 [SMILES: CCOC(=O)/C=C(\C)CC/C=C(\C)CCC=C1CCCCC1] |
| 134 | 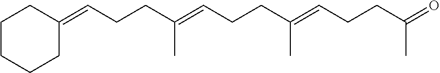 [SMILES: CC(=O)CC/C=C(\C)CC/C=C(\C)CCC=C1CCCCC1] |
| 135 | 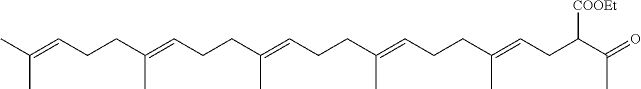 [SMILES: CCOC(=O)C(C/C=C(\C)CC/C=C(\C)CC/C=C(\C)CC/C=C(\C)CCC=C(C)C)C(C)=O] |
| 136 | 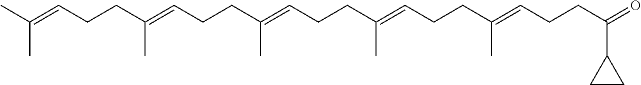 [SMILES: CC(C)=CCC/C(C)=C/CC/C(C)=C/CC/C(C)=C/CC/C(C)=C/CCC(=O)C1CC1] |
| 137 | 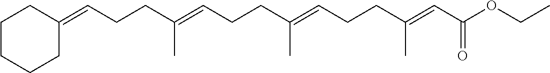 [SMILES: CCOC(=O)/C=C(\C)CC/C=C(\C)CC/C=C(\C)CCC=C1CCCCC1] |
| 138 | 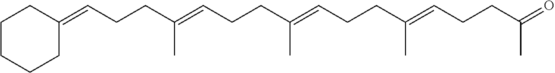 [SMILES: CC(=O)CC/C=C(\C)CC/C=C(\C)CC/C=C(\C)CCC=C1CCCCC1] |
| 139 | 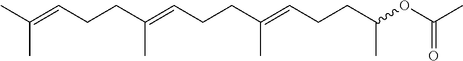 [SMILES: CC(=O)OC(C)CC/C=C(\C)CC/C=C(\C)CCC=C(C)C] |
| 140 | 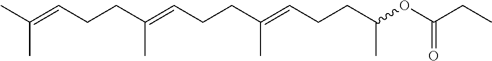 [SMILES: CCC(=O)OC(C)CC/C=C(\C)CC/C=C(\C)CCC=C(C)C] |
| 141 | 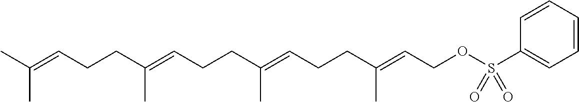 [SMILES: CC(C)=CCC/C(C)=C/CC/C(C)=C/CC/C(C)=C/COS(=O)(=O)c1ccccc1] |
| 142 | 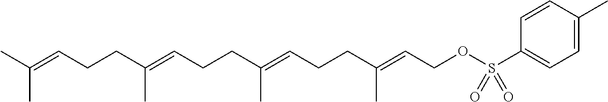 [SMILES: CC(C)=CCC/C(C)=C/CC/C(C)=C/CC/C(C)=C/COS(=O)(=O)c1ccc(C)cc1] |
| 143 | 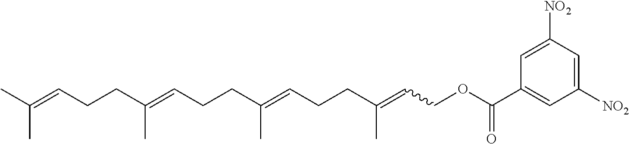 [SMILES: CC(C)=CCC/C(C)=C/CC/C(C)=C/CCC(C)=CCOC(=O)c1cc([N+](=O)[O-])cc([N+](=O)[O-])c1] |
| 144 | 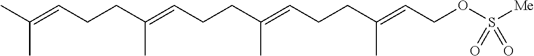 [SMILES: CC(C)=CCC/C(C)=C/CC/C(C)=C/CC/C(C)=C/COS(C)(=O)=O] |
| 145 | 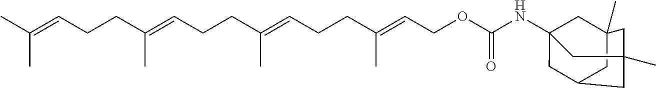 [SMILES: CC(C)=CCC/C(C)=C/CC/C(C)=C/CC/C(C)=C/COC(=O)NC12CC3CC(C)(CC(C)(C3)C1)C2] |
| 146 | 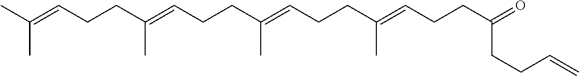 [SMILES: C=CCCC(=O)CC/C=C(\C)CC/C=C(\C)CC/C=C(\C)CCC=C(C)C] |
| 147 | 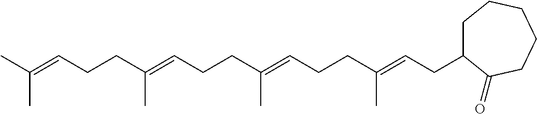 [SMILES: CC(C)=CCC/C(C)=C/CC/C(C)=C/CC/C(C)=C/CC1CCCCCC1=O] |

### Synthesis of GGA Derivatives

Certain methods for making GGA or certain GGA derivatives provided and/or utilized herein are described in PCT publication no. WO 2012/031028 and PCT application no. PCT/US2012/027147, each of which are incorporated herein by reference in its entirety. Other GGA derivatives can be prepared by appropriate substitution of reagents and starting materials, as will be well known to the skilled artisan upon reading this disclosure.

The reactions are preferably carried out in a suitable inert solvent that will be apparent to the skilled artisan upon reading this disclosure, for a sufficient period of time to ensure substantial completion of the reaction as observed by thin layer chromatography, ¹H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure.

The compounds provided and/or utilized in this invention are synthesized, e.g., from a compound of formula (III-A):

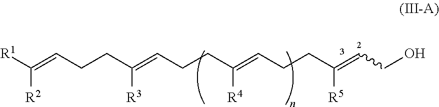

wherein n, R¹-R⁵ and [symbol] are defined as in Formula (I) above, following various well known methods upon substitution of reactants and/or altering reaction conditions as will be apparent to the skilled artisan upon reading this disclosure. The compound of Formula (III-A) is itself prepared by methods well known to a skilled artisan, for example, and without limitation, those described in PCT Pat. App. Pub. No. WO 2012/031028 and PCT Pat. App. No. PCT/US2012/027147 (each supra). An illustrative and non-limiting method for synthesizing a compound of Formula (III-A), where n is 1, is schematically shown below.

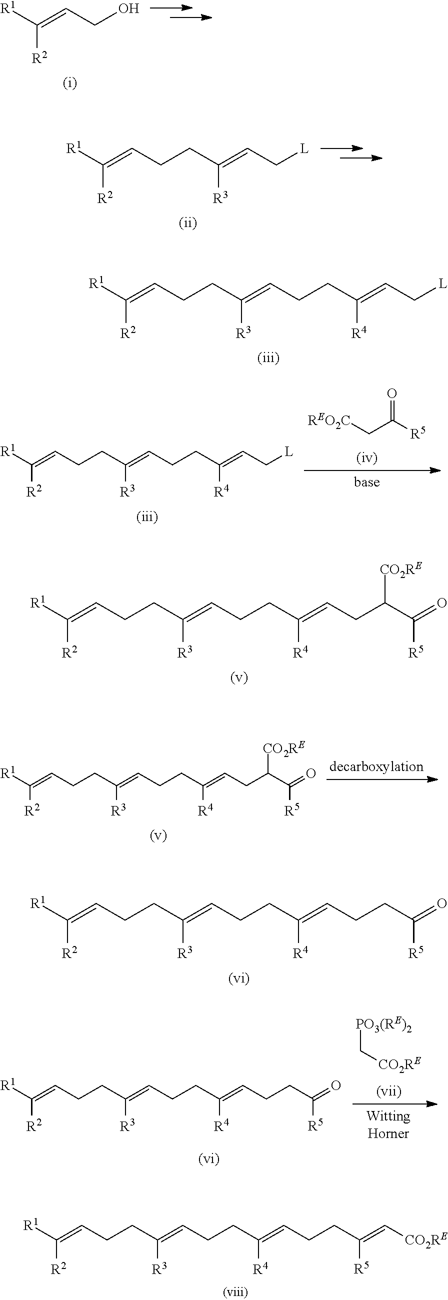

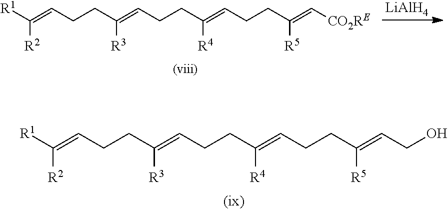

Starting compound (iii), which is synthesized from compound (i) by adding isoprene derivatives as described here, is alkylated with a beta keto ester (iv), in the presence of a base such as an alkoxide, to provide the corresponding beta-ketoester (v). Compound (v) upon alkaline hydrolysis followed by decarboxylation provides ketone (vi). Keto compound (vi) is converted, following a Wittig Horner reaction with compound (vii), to the conjugated ester (viii). Compound (viii) is reduced, for example with LiAlH₄, to provide alcohol (ix).

As will be apparent to the skilled artisan, a compound of Formula (III), where n is 2, is synthesized by repeating the reaction sequence of alkylation with a beta-keto ester, hydrolysis, decarboxylation, Wittig-Horner olefination, and LiAlH₄ reduction.

Certain illustrative and non-limiting synthesis of compounds provided and/or utilized in this invention are schematically shown below. Compounds where Q¹ is —(C═S)— or —SO₂— are synthesized by substituting the carbonyl group of the reactants employed, as will be apparent to the skilled artisan.

R⁶ in the schemes below may also correspond to R³⁰ and R⁵¹ as defined herein. R⁷ in the schemes below may also correspond to R²⁶, R³¹ and R⁵² as defined herein. R⁸ in the schemes below may also correspond to R²⁷, R³² and R⁵³ as defined herein. R⁹ in the schemes below may also correspond to R²⁸, R³³ and R⁵⁴ as defined herein. R¹³ in the schemes below may also correspond to R⁵⁸ as defined herein. R¹⁴ in the schemes below may also correspond to R⁵⁹ as defined herein. R¹⁵ in the schemes below may also correspond to R⁶⁰ as defined herein. R¹⁸ in the schemes below may also correspond to R²⁴, R³⁴ and R⁶⁰ as defined herein. R¹⁹ in the schemes below may also correspond to R²⁵, R³⁵ and R⁶⁴ as defined herein. L is a leaving group as known to one of ordinary skill in the art.

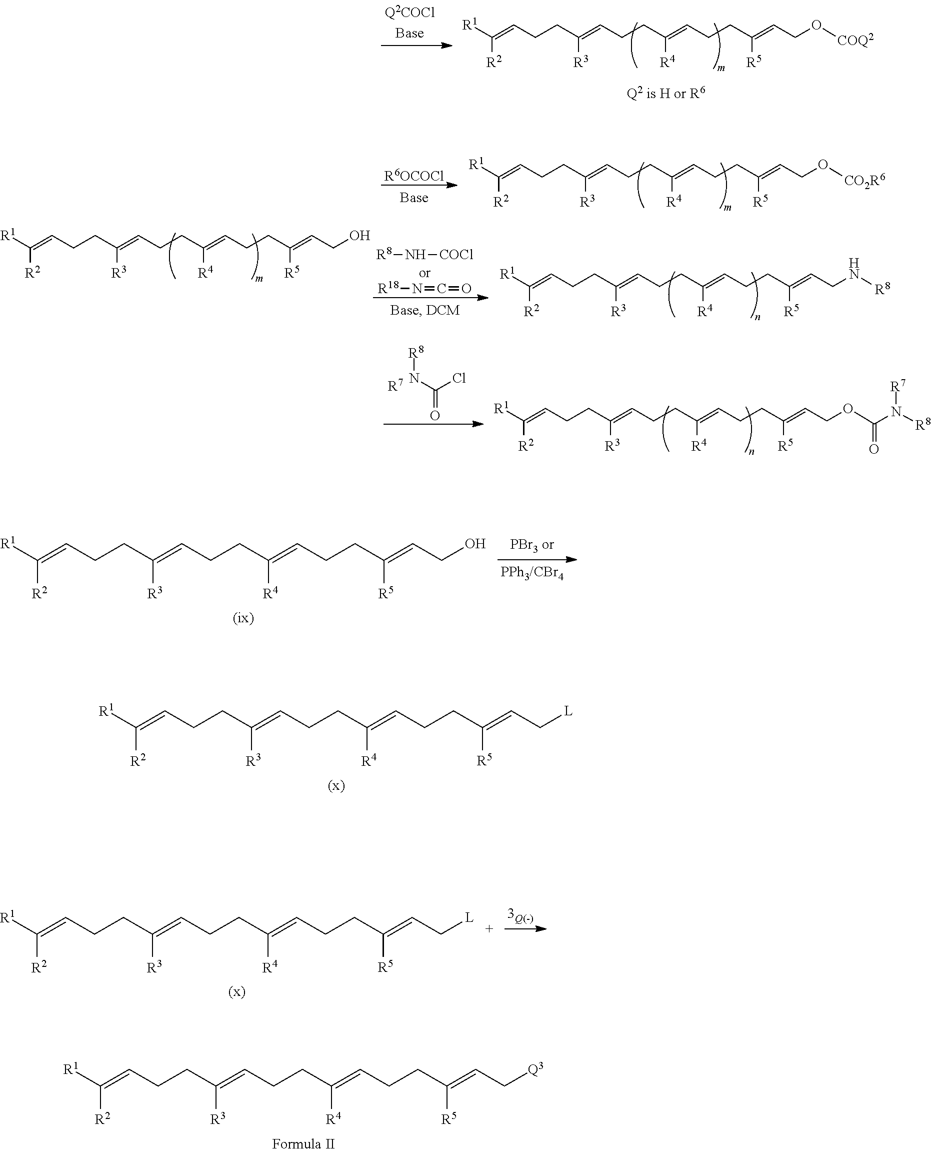

As shown above, R^{E} is alkyl.

Compound (ix) with alcohol functionality is an intermediate useful for preparing the compounds provided and/or utilized in this invention. Compound (x), where L is an R^{s}SO₂— group is made by reacting compound (ix) with R^{s}SO₂Cl in the presence of a base. The transformation of compound (iii) to compound (x) illustrates methods of adding isoprene derivatives to a compound, which methods are suitable to make compound (iii) from compound (i). Intermediate (ix) containing various R¹-R⁵ substituents are prepared according to this scheme as exemplified herein below. The transformation of compound (iii) to compound (x) illustrates methods of adding isoprene derivatives to a compound, which methods are suitable to make compound (iii) from compound (i).

The intermediates prepared above are converted to the compounds provided and/or utilized in this invention as schematically illustrated below:

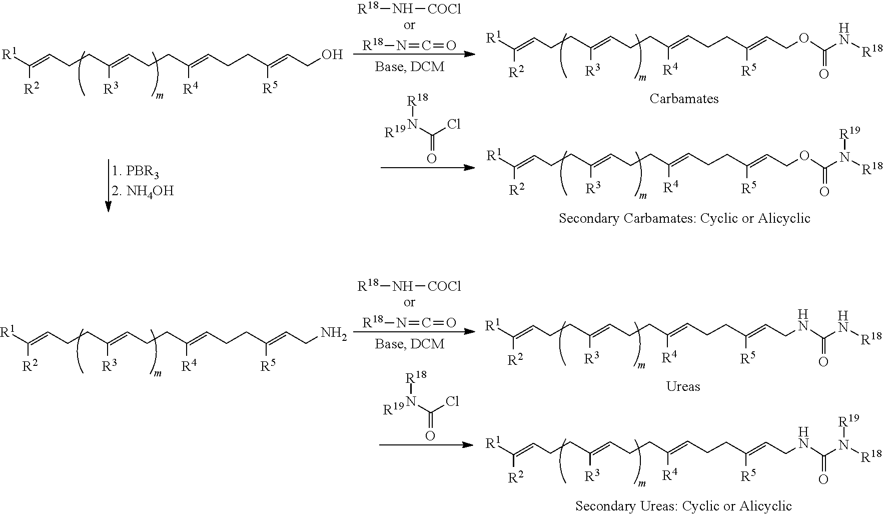

As used herein, for example, and without limitation, m is 0 or 1 and R¹-R⁵ are as defined herein, and are preferably alkyl, or more preferably methyl. Intermediate (ixa), prepared according to the scheme herein above, is converted to amino intermediate (ixb) via the corresponding bromide. Intermediates (ixa) and (ixb) are converted to the compounds provided and/or utilized in this invention by reacting with suitable isocyanates or carbamoyl chlorides, which are prepared by art known methods. The thiocarbamates and thioureas of this invention are prepared according to the methods described above and replacing the isocyanates or the carbamoyl chlorides with isothiocyanates (R¹⁸—N═C═S) or thiocarbamoyl chlorides (R¹⁸—NH—C(═S)Cl or R¹⁸R¹⁹N—C(═S)Cl). These and other compounds provided and/or utilized in this invention are also prepared by art known methods, which may require optional modifications as will be apparent to the skilled artisan upon reading this disclosure. Intermediates for synthesizing compounds provided and/or utilized in this invention containing various R¹-R⁵ substituents are illustrated in the examples section and/or are well known to the skilled artisan.

Certain GGA derivatives provided and/or utilized herein are synthesized as schematically shown below.

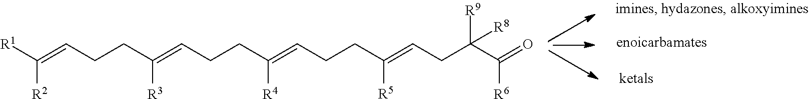

Certain compounds provided and/or utilized herein are obtained by reacting compound (x) with the anion Q(−), which can be generated by reacting the compound QH with a base. Suitable nonlimiting examples of bases include hydroxide, hydride, amides, alkoxides, and the like. Various compounds provided and/or utilized in this invention, wherein the carbonyl group is converted to an imine, a hydrazone, an alkoxyimine, an enolcarbamate, a ketal, and the like, are prepared following well known methods.

Other methods for making the compounds provided and/or utilized in this invention are schematically illustrated below:

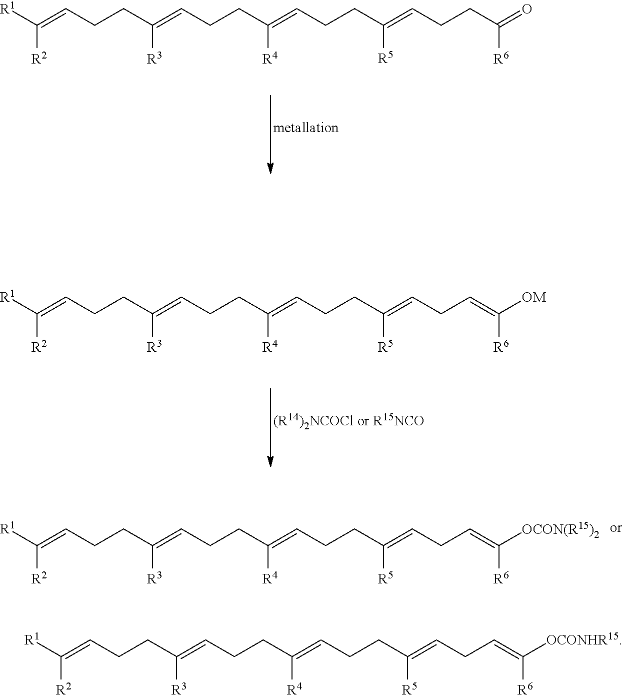

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine (R¹⁴)₂NH with phosgene or an equivalent reagent well known to the skilled artisan.

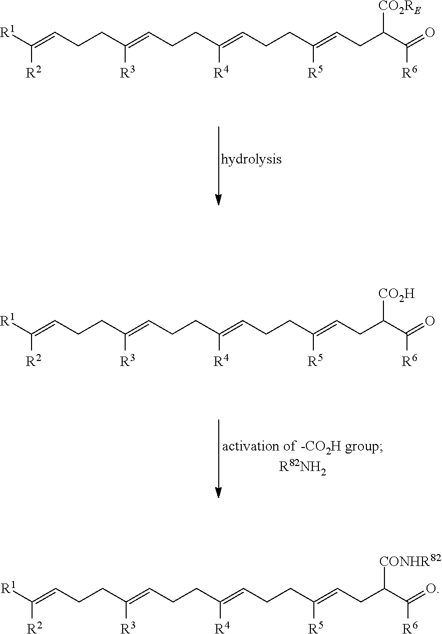

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine.

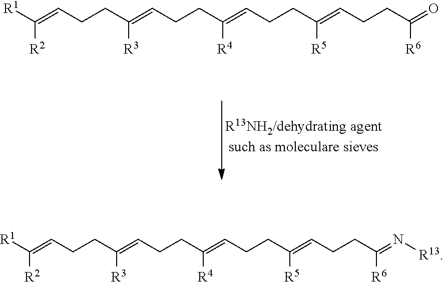

Various other compounds provided and/or utilized in this invention are prepared from the compounds made in the scheme above based on art known methods.

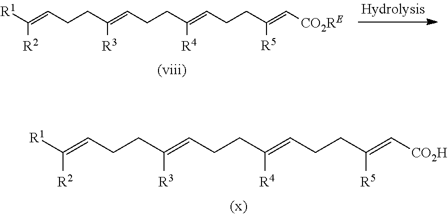

As shown above, R^{F} is alkyl.

The intermediates prepared above are converted to the compounds provided and/or utilized in this invention as schematically illustrated below:

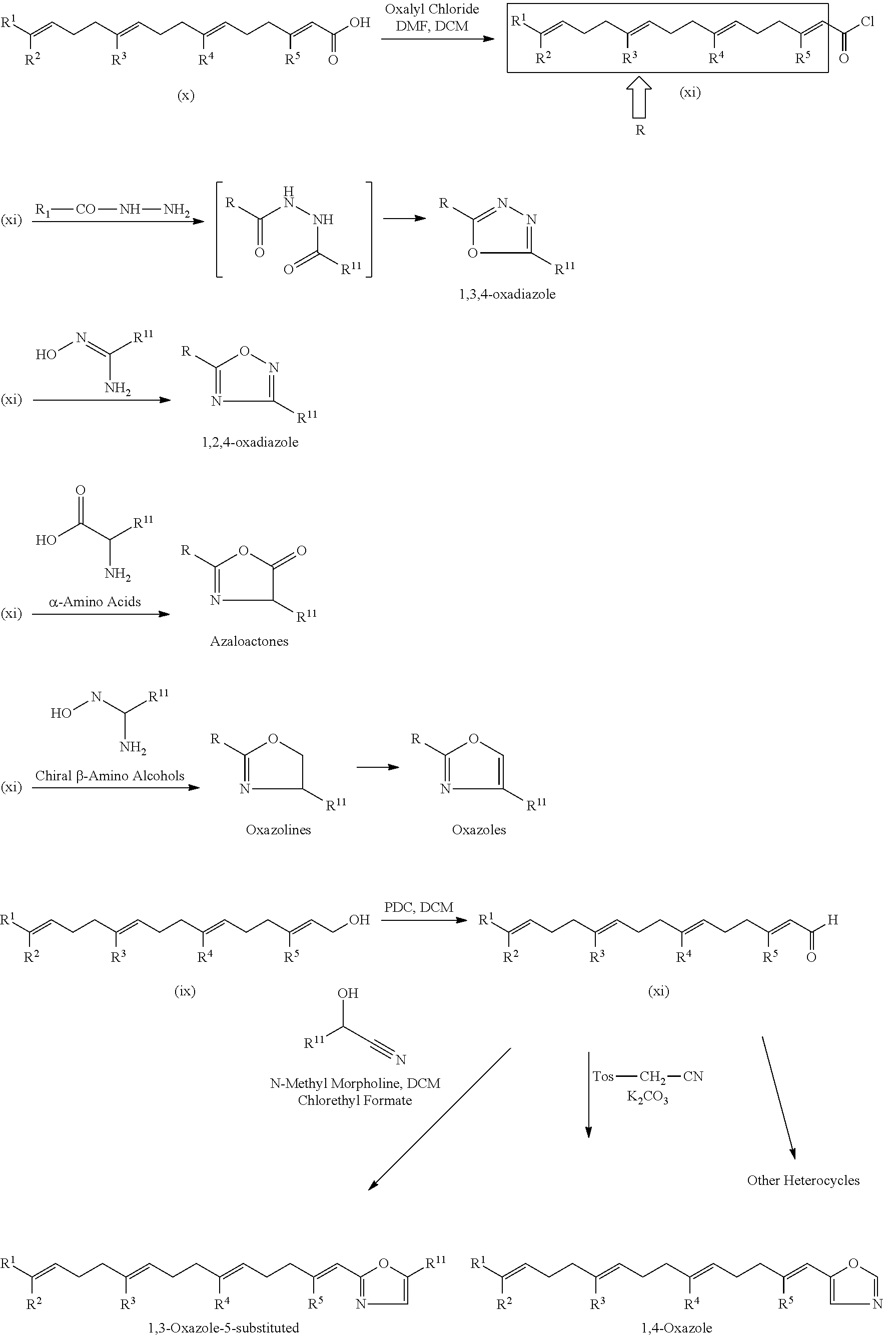

Compound (viii) is hydrolyzed to the carboxylic acid (x), which is then converted to the acid chloride (xi). Compound (xi) is reacted with a suitable nucleophile such as a hydrazide, a hydroxylamine, an amino alcohol, or an amino acid, and the intermediate dehydrated to provide a compound of Formula (IV). Alternatively, the allylic alcohol (ix) is oxidized to the aldehyde (xi), which is then reacted with a cyanohydrin or cyanotosylmethane to provide further compounds provided and/or utilized in this invention.

GGA derivatives provided and/or utilized in this invention can also be synthesized employing art known methods and those disclosed here by alkene-aryl, alkene-heteroaryl, or alkene-akene couplings such as Heck, Stille, or Suzuki coupling. Such methods can use (vi) to prepare intermediate (xii) that can undergo Heck, Stille, or Suzuki coupling under conditions well known to the skilled artisan to provide compounds provided and/or utilized in this invention.

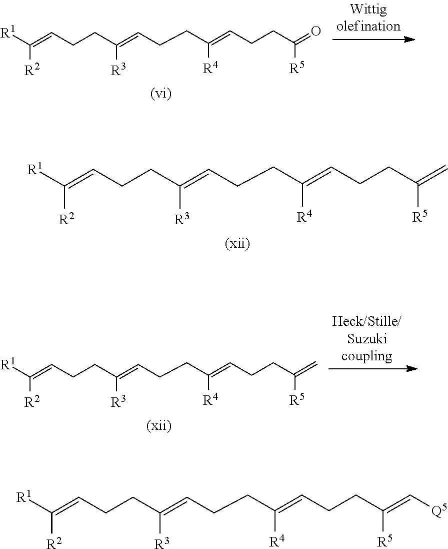

Higher and lower isoprenyl homologs of intermediates (x), (xi), and (xii), which are prepared following the methods disclosed here, can be similarly employed to prepare other compounds provided and/or utilized in this invention.

Compounds provided and/or utilized in this invention are also prepared as shown below

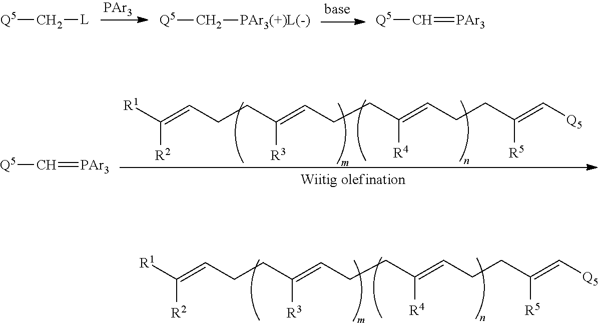

L is a leaving group and Q₅ are as defined herein, Ar is preferably an aryl group such as phenyl, the base employed is an alkoxide such as tertiarybutoxide, a hydride, or an alkyl lithium such as n-butyl lithium. Methods of carrying out the steps shown above are well known to the skilled artisan, as are conditions, reagents, solvents, and/or additives useful for performing the reactions and obtaining the compound of Formula (IV) in the desired stereochemistry.

Other methods for making the compounds provided and/or utilized in this invention are schematically illustrated below:

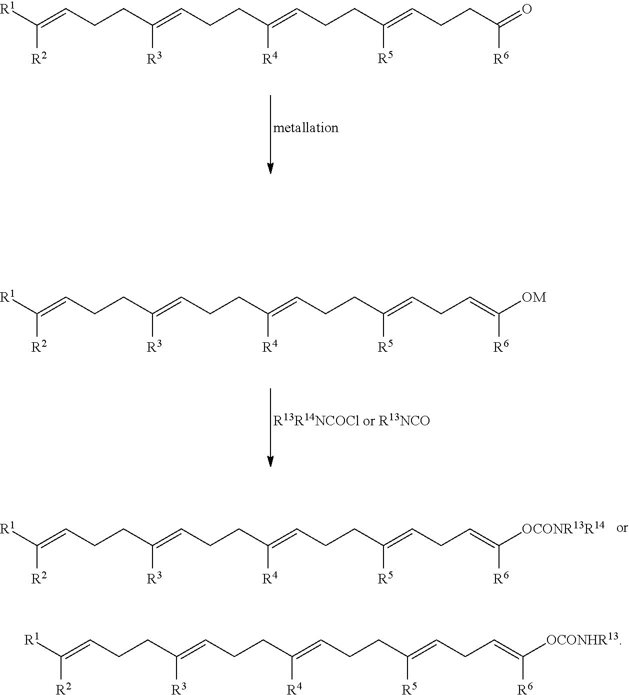

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine R¹³R¹⁴NH with phosgene or an equivalent reagent well known to the skilled artisan.

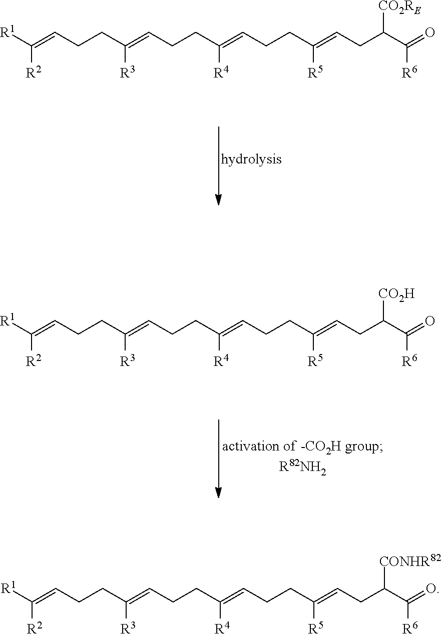

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine. Certain other methods of preparing the conjugates are shown below.

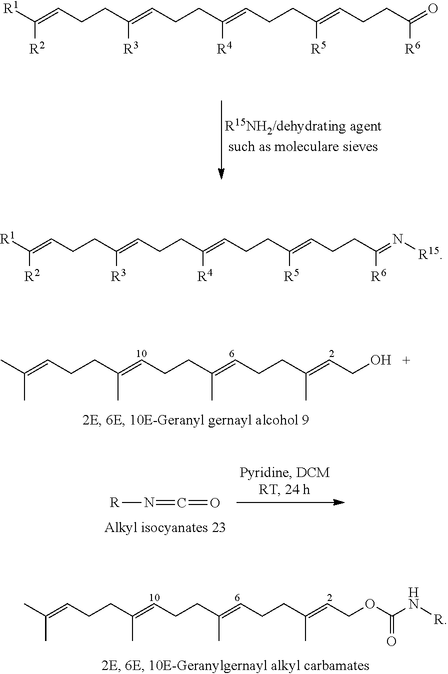

As shown above, R is a memantine or a riluzole residue.
Polyprenyl amine-GGA derivatives can be prepared by reductive amination employing the appropriate polyprenyl aldehyde, a primary or secondary amine and a borohydride reducing agent, as is well known to the skilled artisan. The reaction can be carried out in THF or diethyl ether, optionally in presence of a protic acid, preferably a mild protic acid catalyst.

### Assays

The usefulness of the compounds utilized herein are assayed by a variety of methods. For example, and without limitation, an osteoclast culture for use in screening is a neonatal mouse calvaria assay. Briefly, four days after birth, the front and parietal bones of neonatal mouse pups (e.g., ICR Swiss white mice) are removed by microdissection and split along the sagittal suture. The bones are then incubated in a specified medium, wherein the medium contains either test or control compounds. Following the incubation, the bones are removed from the media, and fixed in 10% buffered formalin, decalcified in EDTA, processed through graded alcohols, and embedded in paraffin wax. Sections of the calvaria are prepared and assessed using histomorphometric analysis of bone formation and bone resorption. Bone changes are measured on sections. Osteoblasts and osteoclasts are identified by their distinctive morphology.

In addition to this assay, the effect of compounds on murine calvarial bone growth can also be tested in vivo. In one such example of this screening assay, young male mice (e.g., ICR Swiss white mice), aged 4-6 weeks are employed, using 4-5 mice per group. Briefly, the test compound or the appropriate control is injected into subcutaneous tissue over the right calvaria of normal mice. The mice are sacrificed (after allowing for bone growth or loss to occur, e.g. on day 14), and net bone growth is measured by histomorphometric means. Bone samples are cleaned from adjacent tissues and fixed in 10% buffered formalin, decalcified, processed through graded alcohols, and embedded in paraffin wax. Sections of the calvaria are prepared, and representative sections are selected for histomorphometric assessment of the effects of bone formation and bone resorption. In one embodiment, sections are measured by using a camera lucida attachment to trace directly the microscopic image onto a digitizing plate. Bone changes are measured on sections over adjacent 1×1 mm fields on both the injected and noninjected sides of calvaria. New bone may be identified by those skilled in the art by its characteristic tinctorial features, and osteoclasts and osteoblasts may be identified by their distinctive morphology or other suitable marker recognized by the skilled artisan. Histomorphometry software (OsteoMeasure, Osteometrix, Inc., Atlanta) can be used to process digitized input to determine cell counts and measure areas or perimeters.

Additional exemplary in vivo assays include dosing assays in intact animals, including dosing assays in acute ovariectomized (OVX) animals and assays in chronic OVX animals. Prototypical dosing in intact animals may be accomplished by subcutaneous, intraperitoneal or oral administration, and may be performed by injection, sustained release or other delivery techniques. The time period for administration of test compound may vary (for instance, 14 days, 28 days, as well as 35 days or longer may be appropriate).

As an example, in vivo oral or subcutaneous dosing assay may be performed as described: In a typical study, 70 three-month-old female Sprague-Dawley rats are weight-matched and divided into treatment groups, with at least several animals in each group. This includes a baseline control group of animals sacrificed at the initiation of the study; a control group administered vehicle only; a PBS or saline-treated control group; and a positive group administered a compound known to enhance net bone formation. Three dosage levels of the test compound are administered to the remaining groups. Test compound, saline, and vehicle are administered (e.g. once per day) for a number of days (for instance at least 14 days, 28 days, or 35 days—wherein an effect is expected in the positive group). All animals are injected with calcein nine days and two days before sacrifice (to ensure proper labeling of newly formed bone). Weekly body weights are determined. At the end of the period of compound administration, the animals are weighed and bled by orbital or cardiac puncture. Serum calcium, phosphate, osteocalcin, and CBCs are determined. Both leg bones (femur and tibia) and lumbar vertebrae are removed, cleaned of adhering soft tissue, and stored in 70% ethanol or 10% formalin for evaluation, for instance as performed by peripheral quantitative computed tomography (pQCT; Ferretti, J, Bone, 17: 353S-364S, 1995), dual energy X-ray absorptiometry (DEXA; Laval-Jeantet A. et al., Calcif Tissue Intl, 56:14-18, 1995, and Casez J. et al., Bone and Mineral, 26:61-68, 1994) and/or histomorphometry. The effect of test compounds on bone remodeling or net bone formation, including bone loss and osteoclast function can thus be evaluated.

Test compounds can also be assayed in acute ovariectomized animals. Such assays may also include an estrogen-treated group as a control. An example of the test in these animals is briefly described: In a typical study, 80 three-month-old female Sprague-Dawley rats are weight-matched and divided into treatment groups, with at least several animals in each group. This includes a baseline control group of animals sacrificed at the initiation of the study; three control groups (sham OVX and vehicle only. OVX and vehicle only, and OVX and PBS only); and a control OVX group that is administered a compound known to block or reduce bone resorption or enhance bone formation (including an anti-resorptive or anabolic compound). Different dosage levels of the test compound are administered to remaining groups of OVX animals.

Since ovariectomy induces hyperphagia, all OVX animals are pair-fed with sham OVX animals throughout the study. Test compound, positive control compound, PBS or saline or vehicle alone is administered orally or subcutaneously (e.g., once per day) for the treatment period. As an alternative, test compounds can be formulated in implantable pellets that are implanted, or may be administered orally, such as by gastric gavage. All animals are injected with calcein nine days and two days before sacrifice. Weekly body weights are determined. At the end of the treatment cycle, the animals blood and tissues are processed as described above.

Test compounds may also be assayed in chronic OVX animals. Briefly, six month old female, Sprague-Dawley rats are subjected to sham surgery (sham OVX), or ovariectomy (OVX) at the beginning of the experiment, and animals are sacrificed at the same time to serve as baseline controls. Body weights are monitored weekly. After approximately six weeks or more of bone depletion, sham OVX and OVX rats are randomly selected for sacrifice as depletion period controls. Of the remaining animals, 10 sham OVX and 10 OVX rats are used as placebo-treated controls. The remaining animals are treated with 3 to 5 doses of test compound for a period of 35 days. As a positive control, a group of OVX rats can be treated with a known anabolic or anti-resorptive agent in this model, such as bisphosphonate, a calcitonin, a calcitriol, an estrogen, selective estrogen receptor modulators (SERM's) and a calcium source, a supplemental bone formation agent parathyroid hormone (PTH) or its derivative (Kimmel et al., Endocrinology, 132: 1577-1584, 1993), PTHRP, a bone morphogenetic protein, osteogenin, NaF, PGE2 agonists, a statin, and a RANK ligand (RANKL), including an osteogenic form of RANKL such as GST-RANKL or other oligomerized form of RANKL. At the end of the experiment, the animals are sacrificed and femurs, tibiae, and lumbar vertebral to 4 are excised and collected. The proximal left and right tibiae are used for pQCT measurements, cancellous bone mineral density (BMD), and histology, while the midshaft of each tibiae is subjected to cortical BMD or histology. The femurs are prepared for pQCT scanning of the midshaft prior to biomechanical testing. With respect to lumbar vertebrae (LV), LV2 are processed for BMD (pQCT may also be performed), LV3 are prepared for undecalcified bone histology, and LV4 are processed for mechanical testing.

In addition, osteoclast cultures, containing macrophages, osteoclast precursors and osteoclasts, can be generated from bone marrow precursors, particularly from bone marrow macrophages and utilized in assessment of compounds for osteoclast modulating activity. Bone marrow macrophages are cultured in 48- or 96-well cell culture dishes in the presence of M-CSF (10 ng/ml), RANKL (100 ng/ml), with or without addition of compound(s) or control(s), and medium changed (e.g. on day 3). Osteoclast-like cells are characterized by staining for tartrate-resistant acid phosphatase (TRAP) activity. In assessing bone resorption, for instance using a pit assay, osteoclasts are generated on whale dentin slices from bone marrow macrophages. After three days of culture to generate osteoclasts, compound(s) or control(s) are added to the culture for two days. At the end of the experiment, cells are TRAP stained and photographed to document cell number. Cells are then removed from the dentin slices with 0.5M ammonium hydroxide and mechanical agitation. Maximum resorption lacunae depth is measured using a confocal microscope (Microradiance, Bio-Rad Laboratories, Hercules, Calif.). For evaluation of pit number and resorbed area, dentin slices are stained with Coumassie brilliant blue and analyzed with light microscopy using Osteomeasure software (Osteometrics, Decatur, Ga.) for quantitation.

In a further method, osteoclast modulating ability of GGA and derivatives can be tested in an in vitro assay utilizing osteoclasts, osteoclast precursor cells or osteoclast-like cells. General protocols for treatment of osteoclasts with a compound are well established and known in the art. For instance, bone marrow macrophages may be utilized to generate osteoclasts in vitro as described herein. It is to be noted that the conditions used will vary according to the cell lines and compound used, their respective amounts, and additional factors such as plating conditions and media composition. Such adjustments are readily determined by one skilled in this art.

### Pharmaceutical Compositions

In further aspects of the invention, a composition for treatment of osteopenia and related conditions or for reducing the negative effects of osteopenia, and related conditions is provided, the composition comprising GGA, preferably all trans GGA, or a GGA derivatives as described herein, or a pharmaceutically acceptable salt thereof, and at least one pharmaceutically acceptable excipient.

Pharmaceutical compositions can be formulated for different routes of administration. Although compositions suitable for oral delivery will probably be used most frequently, other routes that may be used include intravenous, intraarterial, pulmonary, rectal, nasal, vaginal, lingual, intramuscular, intraperitoneal, intracutaneous, transdermal, intracranial, and subcutaneous routes. Other dosage forms include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16^{th} ed., A. Oslo editor, Easton Pa. 1980).

The compositions are comprised of in general, GGA or a GGA derivative in combination with at least one pharmaceutically acceptable excipient. Acceptable excipients are non-toxic, aid administration, and do not adversely affect the therapeutic benefit of the compound of this invention. Such excipients may be any solid, liquid, semi-solid or, in the case of an aerosol composition, gaseous excipient that is generally available to one of skill in the art. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc.

The concentration of the excipient is one that can readily be determined to be effective by those skilled in the art, and can vary depending on the particular excipient used. The total concentration of the excipients in the solution can be from about 0.001% to about 90% or from about 0.001% to about 10%.

In certain preferred embodiments of this invention, there is provided a pharmaceutical composition comprising GGA or a GGA derivative and α-tocopherol. A related embodiment provides for a pharmaceutical composition comprising GGA or a GGA derivative, α-tocopherol, and hydroxypropyl cellulose. In another embodiment, there is provided a pharmaceutical composition comprising GGA or a GGA derivative, α-tocopherol, and gum arabic. In a further embodiment, there is a pharmaceutical composition comprising GGA or a GGA derivative, and gum arabic. In a related embodiment, there is provided GGA or a GGA derivative, gum arabic and hydroxypropyl cellulose.

When α-tocopherol is used alone or in combination with other excipients, the concentration by weight can be from about 0.001% to about 1% or from about 0.001% to about 0.005%, or from about 0.005% to about 0.01%, or from about 0.01% to about 0.015%, or from about 0.015% to about 0.03%, or from about 0.03% to about 0.05%, or from about 0.05% to about 0.07%, or from about 0.07% to about 0.1%, or from about 0.1% to about 0.15%, or from about 0.15% to about 0.3%, or from about 0.3% to about 0.5%, or from about 0.5% to about 1% by weight. In some embodiments, the concentration of α-tocopherol is about 0.001% by weight, or alternatively about 0.005%, or about 0.008%, or about 0.01%, or about 0.02%, or about 0.03%, or about 0.04%, or about 0.05% by weight.

When hydroxypropyl cellulose is used alone or in combination with other excipients, the concentration by weight can be from about 0.1% to about 30% or from about 1% to about 20%, or from about 1% to about 5%, or from about 1% to about 10%, or from about 2% to about 4%, or from about 5% to about 10%, or from about 10% to about 15%, or from about 15% to about 20%, or from about 20% to about 25%, or from about 25% to about 30% by weight. In some embodiments, the concentration of hydroxypropyl cellulose is about 1% by weight, or alternatively about 2%, or about 3%, or about 4%, or about 5%, or about 6%, or about 7%, or about 8%, or about 10%, or about 15% by weight.

When gum arabic is used alone or in combination with other excipients, the concentration by weight can be from about 0.5% to about 50% or from about 1% to about 20%, or from about 1% to about 10%, or from about 3% to about 6%, or from about 5% to about 10%, or from about 4% to about 6% by weight. In some embodiments, the concentration of hydroxypropyl cellulose is about 1% by weight, or alternatively about 2%, or about 3%, or about 4%, or about 5%, or about 6%, or about 7%, or about 8%, or about 10%, or about 15% by weight.

The concentration of GGA or a GGA derivative can be from about 1 to about 99% by weight in the pharmaceutical compositions provided herein. In certain embodiments, the concentration of GGA or a GGA derivative in the pharmaceutical composition is about 5% by weight, or alternatively, about 10%, or about 20%, or about 1%, or about 2%, or about 3%, or about 4%, or about 6%, or about 7%, or about 8%, or about 9%, or about 11%, or about 12%, or about 14%, or about 16%, or about 18%, or about 22%, or about 25%, or about 26%, or about 28%, or about 30%, or about 32%, or about 34%, or about 36%, or about 38%, or about 40%, or about 42%, or about 44%, or about 46%, or about 48%, or about 50%, or about 52%, or about 54%, or about 56%, or about 58%, or about 60%, or about 64%, or about 68%, or about 72%, or about 76%, or about 80% by weight.

In one embodiment, this invention provides sustained release formulations such as drug depots or patches comprising an effective amount of GGA or a GGA derivative. In another embodiment, the patch further comprises gum Arabic or hydroxypropyl cellulose separately or in combination, in the presence of alpha-tocopherol. Preferably, the hydroxypropyl cellulose has an average MW of from 10,000 to 100,000. In a more preferred embodiment, the hydroxypropyl cellulose has an average MW of from 5,000 to 50,000. The patch contains, in various embodiments, an amount of the GGA or a GGA derivative, which is sufficient to maintain a therapeutically effective amount of GGA or a GGA derivative in the plasma for about 12 hours.

Compounds and pharmaceutical compositions of this invention maybe used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

In some embodiments, a compound of this invention can be used as an adjunct to conventional drug therapy of the conditions described herein.

### EXAMPLES

### Inhibition of Osteoclast Differentiation and Activation In Vitro

Osteoclasts are large multinucleated cells derived from the myelomoncitic lineage that adhere to and resorb bone through the local production of the lytic enzymes cathepsin K and tartrate-resistant acid phosphatase (TRAP), which degrade bone protein and mineral content. Osteoclasts can be isolated from animal bones or can be generated from myeloid precursors via differentiation in vitro. Myeloid cells treated in vitro with the cytokines Colony-Stimulating Factor-1 (CSF-1) and receptor activator of nuclear factor kappa-B ligand (RANKL) (Boyle, W. J. et al., Nature 423(6937):337-42, 2003) differentiate into mature osteoclasts that express Cathepsin K and TRAP and are capable of resorbing cortical bone slices. Compounds that are used to treat bone diseases characterized by pathologic bone loss either block osteoclast differentiation and/or osteoclast activation of bone resorption.

### Example 1

### Effect of GGA on Osteoclastogenesis

To determine the effect of geranylgeranylacetone (GGA) on osteoclastogenesis, osteoclast precursors are derived by taking the nonadherent bone marrow cells after an overnight incubation in CSF-1/M-CSF (macrophage colony stimulating factor), and culturing the cells for an additional 4 days with 1,000-2,000 U/ml CSF-1. (Lacey et al., Cell 93, 165-176, 1998). Following 4 days of culture, the adherent cells, which are bone marrow macrophages, can then be exposed to 100 ng/ml RANKL and cultured for 3-5 days. The generation of mature osteoclast can be measured by counting multinucleated TRAP positive cells or by measuring TRAP enzyme activity using histoperoxidase assays as described. Test agents such as GGA and derivatives can be added during this terminal period as well to determine their effects on osteoclast differentiation.

### Example 2

### Effects of GGA and GGA Derivatives on Bone Resorption In Vitro

To assess the effects of GGA and derivatives on bone resorption in vitro one can use the bone pit assay as described by Burgess et al. (J. Cell Biol. 145(3): 527-538, 1999). Osteoclasts can be differentiated on the surface or cortical or dentin bone slices in the presence of CSF-1 and RANKL, then treated with test compounds to look at the impact on bone resorption pit formation as described.

### Example 3

### Inhibition of Osteoclast Function In Vivo by Monitoring Bone Resorption

GGA and derivatives can be tested for their ability to modulate osteoclast function by administering to animals and monitoring bone resorption. One model is to determine the effects on bone resorption of young growing mice as previously described (Schenk et al., Calcif. Tissues Int 38:342-349, 1986; Simonet et al., Cell 89, 308-319, 1997). Young growing mice aged 3-4 weeks, weight range 9.2-15.7 g are divided into groups often mice per group. These mice are injected subcutaneously with saline or test compounds bid for 14 days (5 mg/kg/day). The mice are then radiographed before treatment, at day 7 and on day 14. The mice were sacrificed 24 hours after the final injection. The right femur is then removed, fixed in zinc formalin, decalcified in formic acid and embedded in paraffin. Sections are cut through the mid region of the distal femoral metaphysis and the femoral shaft. Bone density, by histomorphometry, is determined in six adjacent regions extending from the metaphyseal limit of the growth plate, through the primary and secondary spongiosa and into the femoral diaphysis (shaft). Radiographic changes are observed after seven days of treatment to detect evidence of a zone of increased bone density in the spongiosa associated with the growth plates in the GGA treated mice relative to that seen in the controls. Histological changes are observed in the distal femoral metaphysis as shown by increased bone density in a regions 1.1 to 2.65 mm in distance from the growth plate. This is a region where bone is rapidly removed by osteoclast-mediated bone resorption in mice. In these rapidly growing young mice, the increase in bone in this region observed with treatment is consistent with an inhibition of bone resorption.

### Example 4

### Effects of GGA and GGA Derivatives on Bone Loss in Ovariectomized Rats

Effects of GGA and derivatives on bone loss can also be assessed in ovariectomized rats, an animal model for postmenopausal osteoporosis. In this model, typically twelve week old female Fisher rats are ovariectomized (OVX) or sham operated and dual x-ray absorptiometry (DEXA) measurements are made of the bone density in the distal femoral metaphysis. After 3 days recovery period, the animals receive daily injections for 14 days as follows: Ten sham operated animals receive vehicle (phosphate buffered saline); Ten OVX animals receive vehicle (phosphate buffered saline); Six OVX animals receive test compounds; Six OVX animals receive pamidronate (PAM) 5 mg/kg SC as a positive control bisphosphonate; Six OVX animals receive estrogen (ESTR) 40 ug/kg SC. After 7 and 14 days post treatment the animals have bone density measured by DEXA. Two days after the last injection the animals are sacrificed and the right tibia and femur removed for histological evaluation.

The DEXA measurements of bone density will allow detection of a trend to reduce bone density following ovariectomy that is modulated by test compounds and positive controls. The histomorphometric analysis of these animals will confirm bone density increases due to the preservation of cortical bone due to inhibition of osteoclast mediated bone resorption.

All abbreviations for scientific terms used herein have their ordinary scientific meaning as known to the skilled artisan.

1. A composition for treating osteopenia and/or reducing one or more negative effects of bone loss, the composition comprising an effective amount of geranylgeranyl acetone (GGA) or a GGA derivative, and a pharmaceutically acceptable excipient.

2. The composition of claim 1, wherein the GGA or a GGA derivative exists at least 80%, or at least 90%, or at least 95%, or at least 99% in the trans isomer.

3. A method for treating osteopenia and/or reducing one or more negative effects of bone loss comprising administering an effective amount of GGA or a GGA derivative, or a composition of claim 1 to a patient in need thereof.

4. A method for inhibiting loss of bone density in a patient in need thereof comprising administering to the patient an effective amount of GGA or a GGA derivative, or a composition of claim 1.

5. A method for inhibiting bone fracture in a patient at risk thereof which bone fracture arises at least in part from pathological bone loss comprising administering to the patient an effective amount of GGA or a GGA derivative, or a composition of claim 1.

6. The method of claim 5, wherein the bone fracture is fracture of the hip.

7. The method of claim 5, wherein the bone fracture is fracture of the vertebrae.

8. A method for inhibiting bone loss and/or facilitating bone growth in a patient at a risk of loss of bone density, comprising administering to the patient an effective amount of GGA or a GGA derivative, or a composition of claim 1.

9. The method of any one of claims 3-8, wherein the GGA or a GGA derivative exists at least 80%, or at least 90%, or at least 95%, or at least 99% in the trans isomer.
